(19) United States
(12) Patent Application Publication
(10) Pub. No.: US 2024/0159771 A1
(43) Pub. Date: May 16, 2024

(54) SIMPLIFIED BIOSAMPLE PROCESSING FOR LC-MS/MS

(71) Applicant: Laboratory Corporation of America Holdings, Burlington, NC (US)

(72) Inventors: Christopher Michael Shuford, Mebane, NC (US); Russell Philip Grant, Chapel Hill, NC (US); Meghan Norris Bradley, Mebane, NC (US); Patricia Louise Miller Holland, High Point, NC (US); Michael Levandoski, Hillsborough, NC (US); Matthew Lee Francis Crawford, Mebane, NC (US); Bradley Collier, Chapel Hill, NC (US)

(73) Assignee: Laboratory Corporation of America Holdings, Burlington, NC (US)

(21) Appl. No.: 18/477,728

(22) Filed: Sep. 29, 2023

Related U.S. Application Data
(63) Continuation of application No. 16/428,290, filed on May 31, 2019, now abandoned.
(60) Provisional application No. 62/679,133, filed on Jun. 1, 2018.
(60) Provisional application No. 62/679,286, filed on Jun. 1, 2018.
(60) Provisional application No. 62/680,256, filed on Jun. 4, 2018.

(51) Int. Cl. G01N 33/68; G01N 30/06; G01N 30/34; G01N 30/72

(52) U.S. Cl. CPC G01N 33/6848; G01N 30/06; G01N 30/34; G01N 30/7233; G01N 33/6851; G01N 2030/027; G01N 2496/80; G01N 2570/00

(57) ABSTRACT

Disclosed are methods and systems using liquid chromatography/tandem mass spectrometry (LC-MS/MS) for the analysis of endogenous biomarkers isolated from biological samples. In certain embodiments, the samples comprise dried body fluids such as dried plasma.

### RELATED APPLICATIONS

This application is a continuation application of U.S. patent application Ser. No. 16/428,290, filed May 31, 2019, which claims priority to U.S. Provisional Patent Application No. 62/679,133, filed Jun. 1, 2018, U.S. Provisional Patent Application No. 62/679,286, filed Jun. 1, 2018, and U.S. Provisional Patent Application No. 62/680,256, filed Jun. 4, 2018. The disclosures of U.S. Provisional Patent Application Nos. 62/679,133, 62/679,286 and 62/680,256 are incorporated by reference in their entireties herein.

### FIELD OF INVENTION

The presently disclosed subject matter relates to methods and systems for simplified biosample processing for LC-MS/MS. In some cases, the methods and systems may be used for detection of biomolecules including hormones, peptides, proteins, nucleic acids, and lipids.

### BACKGROUND

There is often a need to assay many different types of biomarkers (i.e., analytes) as part of determining the health status of an individual. While plasma and/or blood samples may be accessed by phlebotomy in a medical setting, this can be inconvenient for the patient. Also, with increasing sensitivity provided by improved assay techniques, often only a few drops, rather than an entire tube of blood is required. In addition to standard serum and plasma specimens, it would be helpful to utilize specimens acquired on blood collection devices that can access small amounts of blood and/or plasma.

There is also a need to apply such simplified sample acquisition methods to highly precise quantitative assays, such as liquid chromatography tandem mass spectrometry (LC-MS/MS) assays of biomarkers. Techniques such as LC-MS/MS, which are highly sensitive and precise require unique considerations with respect to standardization and normalization.

### SUMMARY

In some embodiments, the presently disclosed subject matter provides methods and systems for simplified biosample processing for LC-MS/MS assays of such biosamples. The method may be embodied in a variety of ways.

In an embodiment, the disclosure provides a method for determining the presence or amount of at least one biomarker of interest in a biological sample, the method comprising: providing a biological sample believed to contain at least one biomarker of interest wherein the biological sample comprises dried plasma; extracting the at least one biomarker of interest and at least one normalizing marker from the dried plasma sample into a liquid solution; measuring the at least one biomarker of interest in the liquid solution by mass spectrometry; measuring the at least one normalizing marker of interest in the liquid solution by an appropriate analytical technique; and using the ratio of the measurements for the at least one biomarker of interest and the at least one normalizing marker of interest to determine the presence or amount of the at least one biomarker of interest in the biological sample.

Also disclosed are systems for performing the methods of the invention. For example, disclosed is a system for determining the presence and/or amount of a biomarker of interest in a biological sample, the system comprising: a device for providing a test sample comprising dried blood and/or plasma; and a station for analyzing the sample by mass spectrometry to determine the presence or amount of the biomarker of interest in the biological sample. In some embodiments the system may comprise: a station for providing a biological sample believed to contain at least one biomarker of interest wherein the biological sample comprises dried plasma; a station for extracting the at least one biomarker of interest and at least one normalizing marker from the dried plasma sample into a liquid solution; a station for measuring the at least one biomarker of interest in the liquid solution by mass spectrometry; a station for measuring the at least one normalizing marker of interest in the liquid solution by an appropriate analytical technique; and a station for using the ratio of the measurements for the at least one biomarker of interest and the at least one normalizing marker of interest to determine the presence or amount of the at least one biomarker of interest in the biological sample. The system may comprise a station for measuring a normalizing marker by any of the methods disclosed herein. In some embodiments, variations are made to these stations or there may be additional stations. For example, in some cases there may be a station for adding an internal standard and/or a station for partially purifying the biomarker extracted from the plasma (e.g., a station for liquid extraction, dilution, protein precipitation, LC and/or HPLC). Also, in some embodiments, some of the stations may be combined. For example, in an embodiment, the station for extracting the biomarker may include devices for adding the internal standard.

In various embodiments of the system, the biomarker of interest may comprise a peptide or protein (e.g., enzymes and other proteins), a hormone, a cytokine a nucleic acid, a lipid or a protein. Or, other biomarkers may be measured. Also, in some cases where the biomarker of interest is a protein, the sample may be subjected to proteolytic digestion to generate a peptide from the protein. Or, other biomarkers may be measured.

A variety of mass spectrometry techniques may be used. For example, the station for mass spectrometry may comprise a tandem mass spectrometer.

As noted above, the system may comprise a station for chromatographic purification of the biomarker of interest prior to mass spectrometry, as for example by high performance liquid chromatography (HPLC). Thus, in alternate embodiments the mass spectrometry may comprise liquid chromatography tandem mass spectrometry (LC-MS/MS), or 2 dimensional LC-MS/MS.

Also, in certain embodiments, as for example where the biomarker of interest is a peptide, the system may comprise a station for subjecting the sample to protease digestion.

In certain embodiments of both the methods and the systems, the device for providing a biological sample comprises a device to immobilize and separate red blood cells from plasma on a solid substrate, such as filter paper or a card.

The system may comprise a computer such that at least one of the stations is controlled by the computer.

### BRIEF DESCRIPTION OF THE DRAWINGS

Having thus described the invention in general terms, reference will now be made to the following non-limiting drawings, which are not necessarily drawn to scale.

FIG. 1 shows substrates having zones of dried red blood cells and/or dried plasma in accordance with an embodiment of the disclosure.

FIG. 2 shows serial 20 μL samplings from a 180 μL finger stick analyzed for white blood cells (WBC), red blood cells (RBC) and hematocrit (HCT) in accordance with an embodiment of the disclosure.

FIG. 3 shows serial 20 μL samplings from a 200 μL finger stick analyzed for the biomarkers: blood urea nitrogen (BUN), glucose, cholesterol, C-reactive protein (CRP), alanine amino transferase (ALT) and albumin in accordance with an embodiment of the disclosure.

FIG. 4 shows a comparison of the three collection methods (venipuncture, finger stick, and transdermal) across a range of biomarker classes that included cholesterol, C-reactive protein (CRP), glucose, high density lipoprotein (HDL), low density lipoprotein (LDL), prostate-specific antigen (PSA), triglycerides, and thyroid stimulating hormone (TSH) in accordance with an embodiment of the disclosure. For each of the biomarkers, the first bar is diluted plasma obtained by venipuncture, the second bar is diluted plasma obtained by finger stick, and the third bar is diluted plasma obtained by transdermal collection.

FIG. 5 shows the change (i.e., reduction) in interference in an assay for testosterone as plasma separated from red blood cells on a sampling paper in accordance with an embodiment of the disclosure. The top image shows five 9 mm square punches taken from the paper and the bottom plot shows the measured value for testosterone from the dried sample as compared to liquid plasma isolated by venipuncture).

FIG. 6 illustrates that a concentration gradient can occur as plasma migrates across a solid substrate where higher punch numbers correspond to more migration. Also shown in FIG. 6 is the correction for such gradient when the data for testosterone was normalized against another internal biomarker in accordance with an embodiment of the disclosure.

FIG. 7 shows is a graphical representation of a distribution of different potential measures for normalization: refractive index (small dashed), chloride (medium dash), total protein (solid line) and albumin (large dash).

FIG. 8 shows the measures for blood urea nitrogen (BUN) (light gray bars) and potential measures for normalization (dark gray bars) for different punches as blood migrates across a solid substrate. The line overlaid on the bar graphs is the normalized result for BUN.

FIG. 9 shows the measurement performance of a finger stick blood sample on an autoanalyzer immunoassay for testosterone using dried plasma and Cl ion as the normalizer as a scatter plot (upper) and a Bland-Altman plot (lower).

FIG. 10 shows example chromatograms of calibrators, quality controls (QCs), and a finger stick sample measurement for testosterone from dried plasma by LC-MS/MS.

FIG. 11 shows correlation data of CDC Phase I Hormone Standardization samples (left upper) and serum samples (right upper); both sets of spotted samples were normalized with chloride ion. The samples were deposited onto a paper membrane, allowed to migrate and dry prior to extraction. The CDC sample values were compared to the known CDC values (CDC target) and the spotted serum values were compared to measurements from neat serum. The percent bias for the CDC samples and the dried serum samples are shown in the lower left and right graphs respectively.

FIG. 12 shows results from dried plasma from forty (40) finger stick samples measured for testosterone and normalized with chloride ion. The plots depict the accuracy of testosterone measurement for the dried plasma (y-axis) when compared to a venous drawn sample of neat serum (x-axis) (upper plot) and the percent bias (lower plot).

FIG. 13 shows measurement of prostate specific antigen (PSA), sex hormone binding globulin (SHBG), and a determination of free testosterone (Free T) from dried plasma obtained from a finger stick collection in accordance with an embodiment of the disclosure.

FIG. 14 shows a method for analyzing dried plasma (¼ inch diameter punches) for a peptide biomarker of interest in accordance with an embodiment of the disclosure.

FIG. 15 shows an assessment of an analyte peptide (Peptide 1) and an internal standard (IS) as a function of hemoglobin concentration in accordance with an embodiment of the disclosure.

FIG. 16 shows a trypsin digestion time course to generate five different peptides using either liquid plasma or dried plasma in accordance with an embodiment of the disclosure.

FIG. 17 shows stability of peptide measurements from trypsin digestion of dry plasma in accordance with an embodiment of the disclosure.

FIG. 18 shows a system in accordance with an embodiment of the disclosure.

FIG. 19 shows a system for high-throughput proteomic genotyping in accordance with an embodiment of the disclosure.

### DETAILED DESCRIPTION

The presently disclosed subject matter now will be described more fully hereinafter with reference to the accompanying description and drawings, in which some, but not all embodiments of the presently disclosed subject matter are shown. The presently disclosed subject matter can be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the presently disclosed subject matter set forth herein will come to mind to one skilled in the art to which the presently disclosed subject matter pertains having the benefit of the teachings presented in the foregoing descriptions and the associated drawings. Therefore, it is to be understood that the presently disclosed subject matter is not to be limited to the specific embodiments disclosed and that modifications and other embodiments are intended to be included within the scope of the appended claims. Although specific terms are employed herein, they are used in a generic and descriptive sense only and not for purposes of limitation. The disclosure utilizes the abbreviations shown below.

### Abbreviations

APCI=atmospheric pressure chemical ionization
HTLC=high turbulence (throughput) liquid chromatography
HPLC=high performance liquid chromatography
LLE=liquid-liquid extraction
LOQ=limits of quantification
LLOQ=lower limit of quantification
N=number of replicates
SST=system suitability test
ULOQ=upper limit of quantification
2D-LC-MS/MS=two-dimensional liquid chromatography hyphenated to tandem mass spectrometry
(LC)-LC-MS/MS=two-dimensional liquid chromatography tandem hyphenated to mass spectrometry
(LC)-MS/MS=liquid chromatography hyphenated to tandem mass spectrometry

### Definitions

While the following terms are believed to be well understood by one of ordinary skill in the art, the following definitions are set forth to facilitate explanation of the presently disclosed subject matter. Other definitions are found throughout the specification. Unless otherwise defined, all technical and scientific terms used herein have the same meaning as commonly understood by one of ordinary skill in the art to which this presently described subject matter belongs.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g. 1 to 6.1, and ending with a maximum value of 10 or less, e.g., 5.5 to 10. Additionally, any reference referred to as being “incorporated herein” is to be understood as being incorporated in its entirety.

The terms “a”, “an”, and “the” refer to “one or more” when used in this application, including the claims. Thus, for example, reference to “a cell” includes a plurality of such cells, unless the context clearly is to the contrary (e.g., a plurality of cells), and so forth.

As used herein, the term “biomarker” or a “biomarker of interest” is any biomolecule that may provide biological information about the physiological state of an organism. In certain embodiments, the presence or absence of the biomarker may be informative. In other embodiments, the level of the biomarker may be informative. In an embodiment, the biomarker of interest may comprise a peptide, a hormone, a nucleic acid, a lipid or a protein. Or, other biomarkers may be measured.

As used herein, the term “biological sample” refers to a sample obtained from a biological source, including, but not limited to, an animal, a cell culture, an organ culture, and the like. The biological sample may be a body fluid. Suitable samples include blood, plasma, serum, urine, saliva, tear, cerebrospinal fluid, organ, hair, muscle, or other tissue sample. In an embodiment, the sample may comprise dried plasma produced from blood separated on a laminar flow device. For example, in some embodiments, the laminar flow device restricts the flow of red blood cells more than liquid plasma thus separating the liquid blood cells from the plasma.

As used herein, the term “body fluid” refers to a liquid sample obtained from a biological source, including, but not limited to, an animal, a cell culture, an organ culture, and the like. Suitable samples include blood, plasma, serum, urine, saliva, tear, cerebrospinal fluid, organ, hair, muscle, or other tissue sampler other liquid aspirate, all which are capable deposition onto a substrate for collection and drying. In an embodiment, the sample body fluid may be separated on the substrate prior to drying. For example, blood may be deposited onto a sampling paper substrate which limits migration of red blood cells allowing for separation of the blood plasma fraction prior to drying in order to produce a dried plasma sample for analysis. May comprise dried plasma.

As used herein, the terms “individual” and “subject” are used interchangeably. A subject may comprise an animal. Thus, in some embodiments, the biological sample is obtained from a mammalian animal, including, but not limited to a dog, a cat, a horse, a rat, a monkey, and the like. In some embodiments, the biological sample is obtained from a human subject. In some embodiments, the subject is a patient, that is, a living person presenting themselves in a clinical setting for diagnosis, prognosis, or treatment of a disease or condition.

As used herein, the terms “purify” or “separate” or derivations thereof do not necessarily refer to the removal of all materials other than the analyte(s) of interest from a sample matrix. Instead, in some embodiments, the terms “purify” or “separate” refer to a procedure that enriches the amount of one or more analytes of interest relative to one or more other components present in the sample matrix. In some embodiments, a “purification” or “separation” procedure can be used to remove one or more components of a sample that could interfere with the detection of the biomarker of interest, for example, one or more components that could interfere with detection of an analyte by mass spectrometry.

As used herein, the term “finger stick” refers to a method of blood sampling whereby a fingertip is lanced, and the capillary blood is collected.

As used herein the term “transdermal” refers to a method of blood sampling whereby micro-needles puncture the skin surface to access capillary blood within the epidermis or dermis (e.g., the upper arm) and blood is collected through vacuum and/or gravity.

As used herein “venipuncture” refers to a method of blood sampling whereby a needle attached to a syringe is used to puncture a vein.

As used herein, the term “within group variability” or “CVg” refers to coefficient of variability of a measurement between subjects in a group.

As used herein, a “calibrator” is a sample created with known biomarker concentration in a matrix which is ideally but not necessarily free from the biomarker of interest. Calibrators are used to generate dose response curve used to determined concentrations of biomarkers in unknown samples.

As used herein, a “quality control” is a sample with a target concentration range which is used to verify the quality of results from an experiment.

As used herein, “chromatography” refers to a process in which a chemical mixture carried by a liquid or gas is separated into components as a result of differential distribution of the chemical entities as they flow around or over a stationary liquid or solid phase.

As used herein, “liquid chromatography” (LC) means a process of selective retardation of one or more components of a fluid solution as the fluid uniformly percolates through a column of a finely divided substance, or through capillary passageways. The retardation results from the distribution of the components of the mixture between one or more stationary phases and the bulk fluid, (i.e., mobile phase), as this fluid moves relative to the stationary phase(s). “Liquid chromatography” includes reverse phase liquid chromatography (RPLC), high performance liquid chromatography (HPLC) and high turbulence liquid chromatography (HTLC).

As used herein, the term “HPLC” or “high performance liquid chromatography” refers to liquid chromatography in which the degree of separation is increased by forcing the mobile phase under pressure through a stationary phase, typically a densely packed column. The chromatographic column typically includes a medium (i.e., a packing material) to facilitate separation of chemical moieties (i.e., fractionation). The medium may include minute particles. The particles may include a bonded surface that interacts with the various chemical moieties to facilitate separation of the chemical moieties such as the biomarker analytes quantified in the experiments herein. One suitable bonded surface is a hydrophobic bonded surface such as an alkyl bonded surface. Alkyl bonded surfaces may include C-4, C-8, or C-18 bonded alkyl groups, preferably C-18 bonded groups. The chromatographic column may include an inlet port for receiving a sample and an outlet port for discharging an effluent that includes the fractionated sample. In the method, the sample (or pre-purified sample) may be applied to the column at the inlet port, eluted with a solvent or solvent mixture, and discharged at the outlet port. Different solvent modes may be selected for eluting different analytes of interest. For example, liquid chromatography may be performed using a gradient mode, an isocratic mode, or a polytyptic (i.e. mixed) mode. In one embodiment, HPLC may performed on a multiplexed analytical HPLC system with a C18 solid phase using isocratic separation with water: methanol as the mobile phase.

As used herein, the term “analytical column” refers to a chromatography column having sufficient chromatographic plates to effect a separation of the components of a test sample matrix. Preferably, the components eluted from the analytical column are separated in such a way to allow the presence or amount of an analyte(s) of interest to be determined. In some embodiments, the analytical column comprises particles having an average diameter of about 5 μm. In some embodiments, the analytical column is a functionalized silica or polymer-silica hybrid, or a polymeric particle or monolithic silica stationary phase, such as a phenyl-hexyl functionalized analytical column.

Analytical columns can be distinguished from “extraction columns,” which typically are used to separate or extract retained materials from non-retained materials to obtained a “purified” sample for further purification or analysis. In some embodiments, the extraction column is a functionalized silica or polymer-silica hybrid or polymeric particle or monolithic silica stationary phase, such as a Poroshell SBC-18 column.

The term “heart-cutting” refers to the selection of a region of interest in a chromatogram and subjecting the analytes eluting within that region of interest to a second separation, e.g., a separation in a second dimension.

The term “electron ionization” as used herein refers to methods in which an analyte of interest in a gaseous or vapor phase interacts with a flow of electrons. Impact of the electrons with the analyte produces analyte ions, which may then be subjected to a mass spectrometry technique.

The term “chemical ionization” as used herein refers to methods in which a reagent gas (e.g. ammonia) is subjected to electron impact, and analyte ions are formed by the interaction of reagent gas ions and analyte molecules.

The term “field desorption” as used herein refers to methods in which a non-volatile test sample is placed on an ionization surface, and an intense electric field is used to generate analyte ions.

The term “matrix-assisted laser desorption ionization,” or “MALDI” as used herein refers to methods in which a non-volatile sample is exposed to laser irradiation, which desorbs and ionizes analytes in the sample by various ionization pathways, including photo-ionization, protonation, deprotonation, and cluster decay. For MALDI, the sample is mixed with an energy-absorbing matrix, which facilitates desorption of analyte molecules.

The term “surface enhanced laser desorption ionization,” or “SELDI” as used herein refers to another method in which a non-volatile sample is exposed to laser irradiation, which desorbs and ionizes analytes in the sample by various ionization pathways, including photo-ionization, protonation, deprotonation, and cluster decay. For SELDI, the sample is typically bound to a surface that preferentially retains one or more analytes of interest. As in MALDI, this process may also employ an energy-absorbing material to facilitate ionization.

The term “electrospray ionization,” or “ESI,” as used herein refers to methods in which a solution is passed along a short length of capillary tube, to the end of which is applied a high positive or negative electric potential. Upon reaching the end of the tube, the solution may be vaporized (nebulized) into a jet or spray of very small droplets of solution in solvent vapor. This mist of droplet can flow through an evaporation chamber which is heated slightly to prevent condensation and to evaporate solvent. As the droplets get smaller the electrical surface charge density increases until such time that the natural repulsion between like charges causes ions as well as neutral molecules to be released.

The term “Atmospheric Pressure Chemical Ionization,” or “APCI,” as used herein refers to mass spectroscopy methods that are similar to ESI, however, APCI produces ions by ion-molecule reactions that occur within a plasma at atmospheric pressure. The plasma is maintained by an electric discharge between the spray capillary and a counter electrode. Then, ions are typically extracted into a mass analyzer by use of a set of differentially pumped skimmer stages. A counterflow of dry and preheated N₂ gas may be used to improve removal of solvent. The gas-phase ionization in APCI can be more effective than ESI for analyzing less-polar species.

The term “Atmospheric Pressure Photoionization” (“APPI”) as used herein refers to the form of mass spectroscopy where the mechanism for the photoionization of molecule M is photon absorption and electron ejection to form the molecular M+. Because the photon energy typically is just above the ionization potential, the molecular ion is less susceptible to dissociation. In many cases it may be possible to analyze samples without the need for chromatography, thus saving significant time and expense. In the presence of water vapor or protic solvents, the molecular ion can extract H to form MH+. This tends to occur if M has a high proton affinity. This does not affect quantitation accuracy because the sum of M+ and MH+ is constant. Drug compounds in protic solvents are usually observed as MH+, whereas nonpolar compounds such as naphthalene or testosterone usually form M+(see e.g., Robb et al., 2000, Anal. Chem. 72(15): 3653-3659).

The term “inductively coupled plasma” as used herein refers to methods in which a sample is interacted with a partially ionized gas at a sufficiently high temperature to atomize and ionize most elements.

The term “ionization” and “ionizing” as used herein refers to the process of generating an analyte ion having a net electrical charge equal to one or more electron units. Negative ions are those ions having a net negative charge of one or more electron units, while positive ions are those ions having a net positive charge of one or more electron units.

The term “desorption” as used herein refers to the removal of an analyte from a surface and/or the entry of an analyte into a gaseous phase.

As used herein, the term “hemolyzed” refers to the rupturing of the red blood cell membrane, which results in the release of hemoglobin and other cellular contents into the plasma or serum and the term “lip emic” refers to an excess of fats or lipids in blood.

As used herein, “liquid plasma” is plasma that is obtained from drawing blood from a patient and that is separated from the red blood cells but that remains in a liquid state. Liquid plasma is generally obtained from subjects by phlebotomy or venipuncture.

As used herein, “dried plasma” is plasma that has been allowed to dry. Dried plasma may be produced following separation from red blood cells by migration of the plasma through pores of a solid substrate which restrict migration of cells as is described in more detail herein.

As used herein, the term “punch” corresponds to a portion of a dried substrate that has a sample of dried blood or plasma on the surface. For example, in an embodiment, the punch is a 9 mm² portion of a filter paper or other sampling paper or solid substrate. Or, the punch may be a circle, or another shape as shown herein.

As used herein, a “sampling paper” or “filter” or “filter membrane” or “laminar flow paper” or “laminar flow device” are terms used interchangeably to refer to a solid substrate for the collection of dried blood and plasma may comprise a filter paper or membrane onto which blood can be spotted and that allows for the migration of the plasma away from the red blood cells to produce a region that is substantially plasma and that upon drying, provides a sample of dried plasma.

As used herein, a “genotype” is the DNA sequence of the two alleles present in a gene that may encode a protein sequence.

As used herein a “surrogate peptide” is a peptide derived from a protein and that provides sequence information about the protein. As used herein, the terms “variant specific surrogate peptide” and/or “allele specific surrogate peptide” and/or “genotype specific surrogate peptide” is a peptide derived from a protein and that has a unique amino acid sequence directly attributable to the DNA sequence of the gene that encodes for the protein. Determination of the sequence of the allele specific surrogate peptide can be used to infer the genotype of at least one allele of the gene. Thus, as used herein, an “allele specific surrogate peptide” is a peptide that provides sequence information about the allele which encodes the protein. For example, for ApoL1, a wild-type surrogate peptide indicates that the protein is derived from a wild-type allele, whereas a G1 surrogate peptide indicates that the protein is derived from the G1 allele, and a G2 surrogate peptide indicates that the protein is derived from the G2 allele.

As used herein, a “common surrogate” peptide or “qualifying peptide” or ‘qualifying surrogate peptide” is a peptide that has a unique amino acid sequence that does not vary when the DNA sequence at a locus of interest may vary. Thus, as used herein, a “common surrogate peptide” or “qualifying peptide” comprises a peptide sequence that is common to the wild-type allele as well as all of the alleles being interrogated. Determination of the sequence of the common surrogate peptide will not vary with changes in the genotype at the locus of interest, and thus can be used as internal controls to differentiate a true negative signal from a sample processing error.

As used herein, a “protein variant” is a protein that has an amino acid sequence that is different from the most common or wild-type sequence.

As used herein, a “proteomic profile” is a profile of surrogate peptides that can be used to determine the genotype of an individual at a locus of interest.

Simplified Biosample Processing for LC-MS/MS

The presently disclosed subject matter relates to methods and systems for simplified biosample processing for LC-MS/MS. In some cases, the methods and systems are particularly suited for proteomic analysis. The present invention may be embodied in a variety of ways.

Methods of Simplified Biosample Processing for LC-MSIMS

In one embodiment, the present invention comprises a method for determining the presence or amount of at least one biomarker of interest in a biological sample, the method comprising: providing a biological sample believed to contain at least one biomarker of interest wherein the biological sample comprises a body fluid; and analyzing the chromatographically separated at least one biomarker of interest by mass spectrometry to determine the presence or amount of the at least one biomarker of interest in the sample. In an embodiment, the method may comprise chromatographically separating the at least one biomarker of interest from other components in the sample.

For example, in an embodiment the method may comprise providing a biological sample believed to contain at least one biomarker of interest wherein the biological sample comprises dried plasma; extracting the at least one biomarker of interest and at least one normalizing marker from the dried plasma sample into a liquid solution; measuring the at least one biomarker of interest in the liquid solution by mass spectrometry; measuring the at least one normalizing marker of interest in the liquid solution by an appropriate analytical technique; and using the ratio of the measurements for the at least one biomarker of interest and the at least one normalizing marker of interest to determine the presence or amount of the at least one biomarker of interest in the biological sample. Or body fluid other than plasma may be used.

In an embodiment, the biomarker of interest may comprise a peptide or protein (e.g., enzymes and other proteins), a hormone, a cytokine a nucleic acid, a lipid or a protein. Or, other biomarkers may be measured. Also, in some cases where the biomarker of interest is a protein, the sample may be subjected to proteolytic digestion to generate a peptide from the protein.

The normalization marker may, in certain embodiments, correct for variation in sample volume (i.e., the amount of dried plasma on the solid support). Additionally, the normalization marker may, in certain embodiments, correct for variation in the concentration of the plasma (and biomarkers therein) on the solid support or other interface used to isolate the plasma (e.g., as the plasma migrates from the whole blood cells across the surface of a filter paper or other sampling support).

The normalization marker is, in certain embodiments, selected specifically for the LC-MS/MS assay of the biomarker of interest. For example, U.S. Pat. No. 7,611,670, incorporated by reference in its entirety herein, describes the use of sodium and chloride as normalization markers for various biomarkers such as cholesterol, triglycerides, high density lipoprotein (HDL), low density lipoprotein (LDL), and alanineamino transferase (ALT) but does not describe the use of LC-MS/MS as the analytical technique. Also, U.S. Pat. No. 6,040,135, also incorporated by reference in its entirety herein, describes the use of hemoglobin as an estimate of blood volume.

In an embodiment, the normalizing marker is also present in the biological sample. The normalizing marker should be a compound that has a low between subject biologic variation (CVg). In an embodiment, the test group may be humans. Or, the test group may be animals or mammals. Or, the test group may be a more narrowly defined group (e.g., females, males, people under or over a certain age, people of a certain ethnicity or race, certain types of animals such as dogs or cats, and the like). In certain embodiments the normalizing marker has a CVg of less than 10.0%, or less than 5.0%, or less than 2.0% or less than 1.0%. In an embodiment, the CVg of the normalizing marker is less than the CVg of the biomarker of interest. For example, in various embodiments, the CVg of the normalizing marker is at least 10-fold, or at least 5-fold- or at least 2-fold less than the CVg of the biomarker of interest. It will be understood that the overall analytical precision for the assay will not be better than (i.e., have a lower variability) the CVg and as such, the marker chosen for normalization may be based, in part, on the nature of the assay. Example normalization markers that may be used are shown in Table 1. Or, other normalization markers may be used. Thus, in some cases, the normalizing marker is chloride or serum albumin present in the blood sample used to produce the dry plasma.

TABLE 1

| Normalizing Marker | CVg (%) |
|---|---|
| Thyroxine binding globulin (TBG) | 0.06 |
| Refractive Index | 0.2 |
| Sodium | 0.7 |
| Specific Gravity | 1.0 |
| Chloride | 1.5 |
| Calcium ions (Ca2+) | 1.9 |
| Lactate Dehydrogenase 2 (LDH2) | 2.4 |
| Calcium | 2.5 |
| Transferrin | 4.3 |
| Total Protein | 4.7 |
| Serum Albumin | 4.75 |
| Sodium Bicarbonate | 4.8 |
| Super Oxide Dismutase | 4.9 |

In certain embodiments, the biomarker and normalizing marker are extracted into a liquid solution from the dried plasma sample using a protein containing solution, such as bovine serum albumin dissolved in water or buffer. In other embodiments, the biomarker and normalizing marker are extracted from the dried plasma into a liquid solution using a water, aqueous buffer, non-aqueous solvent, or mixtures thereof. Or, other extraction buffers (or water) may be used.

For example, it is expected that concentration of the biomarker present in a dry plasma may not be uniformly distributed across the laminar flow device due to, for example, differences in the hematocrit content of the blood deposited onto the device or heterogeneity in the laminar flow substrate. Further, the volume of liquid blood deposited onto the device need not be known and corresponding volume of plasma analyzed in dry form may not be known. To correct for uncertainty in volume and biomarker concentration distribution, a normalizing marker with an expected concentration may be measured to normalize for this source of variation. For example, if the measured concentration of the normalizing marker in the extracted dry plasma solution is 30% lower than its expected concentration, it may be assumed that the recovery of the biomarker concentration in the extracted dry plasma solution is also 30% lower than its true concentration allowing for correction of the recovery loss. As such, it is ideal that the normalizing marker have 1) a tightly controlled expected concentration 2) similar distribution in the dry plasma on the substrate as the biomarker(s) of interest, and 3) similar extraction recovery from the dry plasma into the liquid solution. Further, the analytical measurement of the normalizing marker should ideally have little analytical variance in order to contribute little variance to the determination of the biomarker concentration. In some instances, it may be appropriate to determine the concentration of the biomarker in the dry plasma, or an extract thereof, as an amount of biomarker per unit of normalizing marker (e.g., nanogram of testosterone per millimole of chloride), rather than using measurement of the normalizing marker to correct for recovery and volume to produce a biomarker concentration in units of amount of biomarker per unit volume of plasma (i.e., nanogram of testosterone per deciliter of plasma).

In addition to a normalizing marker, an internal standard may be used. The internal standard may be added to correct for variability in the assay steps, as opposed to the normalization marker which is used to correct for sampling variability. Thus, in certain embodiments, the internal standard is added at the beginning of the analytical procedure. Or, an internal standard may be added at different points in the method. For example, the internal standard could be added directly to the blood sample, or the internal standard could be added at a later step in the analytical procedure.

For example, in certain embodiments, the solution used for extraction of the dried plasma may contain an internal standard for the biomarker to correct for variation (imprecision) during subsequent extraction and measurements. In some cases, the internal standard may be a stable isotope labeled analogue of the biomarker. Additionally and/or alternatively, the solution used for extraction of the dried plasma may contain an internal standard for the normalizing marker. In some cases, the internal standard may be a stable isotope labeled analogue of the normalizing marker. Or, other internal standards may be used. For example, internal standards may include, but are not limited to, molecules labeled with other types (i.e., other than an isotope) of detectable moieties (e.g., fluorophores, radiolabels, organic side chains (e.g., a methyl group), nucleic acid molecules with a single base substitution for a nucleic acid biomarker, internal or surrogate peptides for a peptide or protein biomarker, stereoisomers e.g., for hormone and other small molecule biomarkers.

Also, in some embodiments at least one of a quality control and/or a calibrator for measurement of the biomarker is added to the sampling paper solid substrate not containing dried plasma. Such calibrators and quality controls may be used to determine unknown concentration and assess quality of the extraction and measurement processes.

In certain embodiments, the analytical technique used to measure both the biomarker and the normalizing marker may be mass spectrometry. In other embodiments, the analytical technique used to measure the biomarker may be mass spectrometry, but the analytical technique used to measure the normalizing marker may not be mass spectrometry. For example, the normalizing marker may be measured by immunometric methods, colorimetric methods, electrochemical methods, or fluorometric methods. Or, the analytical technique used to measure the biomarker may not be mass spectrometry (but may be one of the other methods disclosed herein), but the analytical technique used to measure the normalizing marker may be mass spectrometry.

In alternate embodiments the mass spectrometry is tandem mass spectrometry, liquid chromatography tandem mass spectrometry (LC-MS/MS), or 2 dimensional LC-MS/MS.

In certain embodiments, the sample is subjected to a purification step prior to mass spectrometry. For example, the purification step may comprise liquid-liquid extraction of the sample, protein precipitation or dilution of the sample prior to mass spectrometry. In one embodiment, the sample is diluted into a solvent or solvent mixture that may be used for LC and/or MS (e.g., LC-MS/MS or 2D-LC-MS/MS).

Additionally and/or alternatively, the purification step may comprise liquid chromatography, such as high performance liquid chromatography (HPLC). In some cases, the chromatography comprises extraction and analytical liquid chromatography. Additionally and/or alternatively, high turbulence liquid chromatography (HTLC) (also known as high throughput liquid chromatography) may be used.

The present disclosure provides a simplified method of biosample processing using dried plasma that has been isolated using plasma separation strips. Such collection devices provide a metering mechanism that spots a known volume of heparinized blood onto plasma separation strips. This automation is intended to provide an easier sampling mechanism for the patient. Dried blood is an alternate specimen collection process that utilizes a finger stick and a plasma separator strip instead of venipuncture collection of serum or plasma tubes. The functional core of the collection strip is a specialized blood separator material that restricts the migration of cells from the application site while allowing the lateral flow of plasma. This selective migration separates the cells and plasma within the lateral flow material similar to the separation obtained from the centrifugation of a serum separator tube. Dried plasma from a standardized punched section of the separation material can be analyzed in place of liquid plasma or serum using established laboratory procedures.

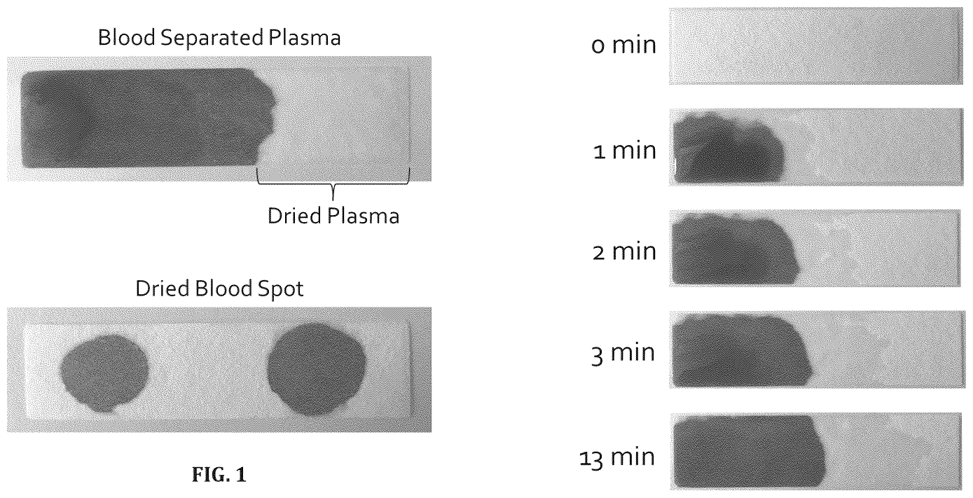

FIG. 1 shows a substrate having zones of dried red blood cells and dried plasma isolated using a sampling paper comprising plasma separated from dried blood as compared to a paper having a dried blood spot, but that does not have the plasma separated from the blood (see FIG. 1 left panel, top and bottom, respectively). The right side of FIG. 1 shows the extent of separation with time. Although dried blood spots are commonly used specimens in clinical analyses, the analysis may suffer from interferences derived from red blood cells (e.g., hemoglobin). Laminar flow paper may be used to separate the plasma fraction of blood deposited from the cellular components due to differential migration of the plasma and cells through the paper, resulting in a cell-free plasma fraction that can be dried and assayed.

The zone of dried plasma can then be isolated, e.g., by punching out small sections that are generally circular although other shapes convenient to the assay format may be used. For example, for 96 well microtiter plates, it was found that circular shaped punch outs of about ¼ inch diameter could be transferred to individual wells for subsequent processing steps. For example, in some cases, extraction may be followed by supported liquid extraction.

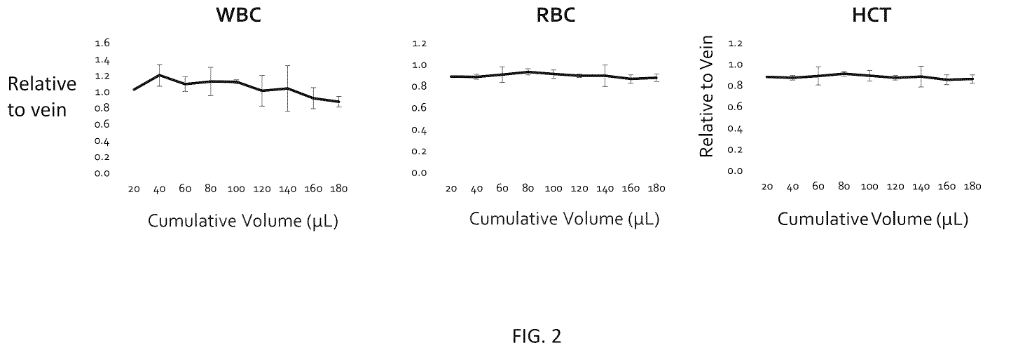

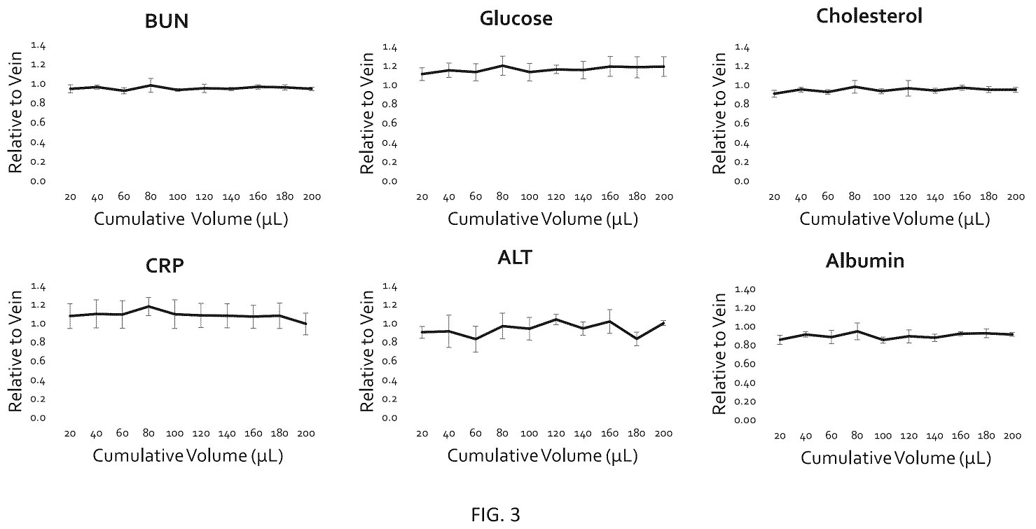

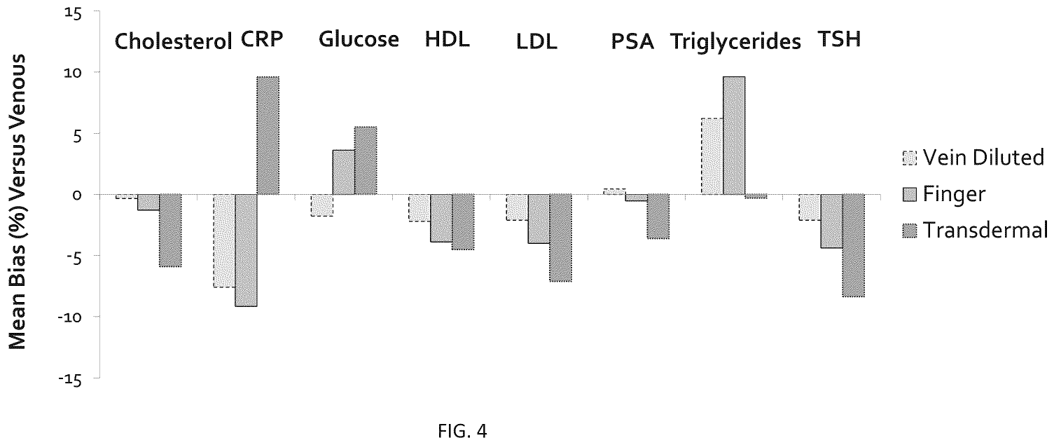

In certain embodiments, the sample is characterized for sampling bias and the results compared to other types of sampling. For example, in certain embodiments, the finger stick sample is reproducible for both types of blood components such as red blood cells (RBC), white blood cells (WBC) and hematocrit (HCT) (FIG. 2) and various biomarkers such as, but not limited to, cholesterol, C-reactive protein (CRP), glucose, high density lipoprotein (HDL), low density lipoprotein (LDL), prostate specific antigen (PSA), triglycerides, and thyroid stimulating hormone (TSH) (FIG. 3) regardless of whether the aliquot used is part of the early sampling (i.e., the volume to first enter the sampling tube used for the finger stick) or is blood collected at the end of the sampling. In certain embodiments, the mean bias for a finger stick sample is less than or equal to the desirable (<4.1%) and/or minimal (<6.2%) mean bias (see e.g., Ricos C. et al., Scand J Clin Lab Invest 1999; 59:491-500). Also, in certain embodiments, dilution of finger stick samples does not lead to untoward negative (or positive) bias (FIG. 4). For example, in an embodiment, the mean bias as compared to venipuncture plasma is ≤20%, or ≤15%, or ≤10%, or ≤8%, or ≤5%, or ≤3%, or ≤2%, or ≤1% (FIG. 4).

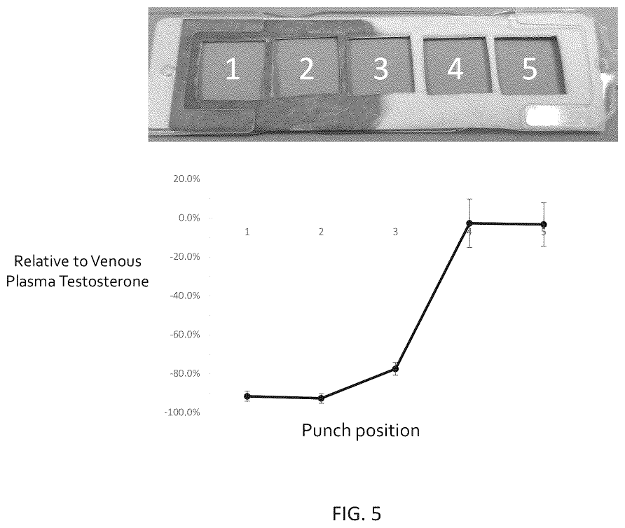

In an embodiment, the use of dried plasma, as compared to dried blood, corrects for any interfering compounds (such as hemoglobin) that are present in blood. For example, where the biomarker is measured by LC-MS/MS, hemoglobin may interfere with the detection of a biomarker of interest in two significant ways. First, at higher concentrations of hemoglobin an isobaric interferent may be observed in SRM MS/MS transitions, which may confound interpretation of specimens. Second, a matrix effect may be observed which can result in lower analytical response. Or other types of interferences may occur depending upon the assay used for either the biomarker or the normalization marker. Thus, as shown in FIG. 5, there may be a reduction in interference in an assay for testosterone as plasma separated (from punch 1 to punch 5 or left to right) from red blood cells on a sampling paper.

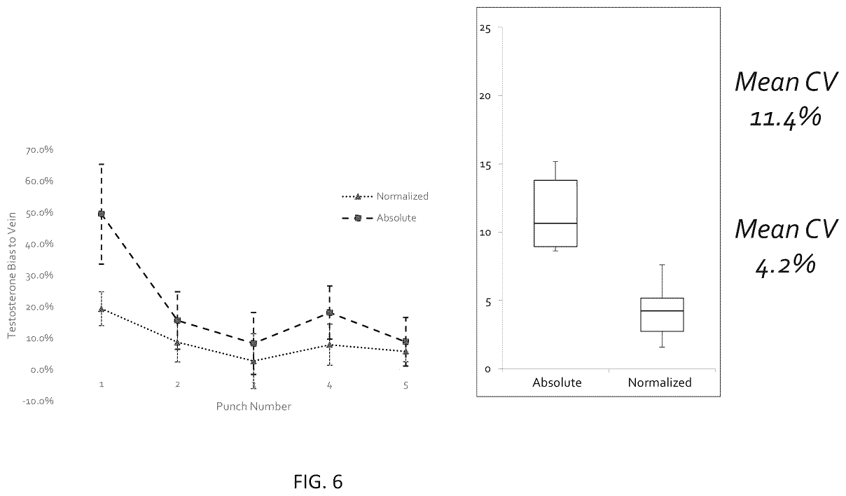

As discussed herein, normalization markers may be used to correct for sampling inconsistences due to for example, a larger or smaller blood sample being applied to the sampling paper and/or the dilution of the biomarker(s) as the sample migrates (e.g., from punch 1 to punch 5) across the sampling paper to allow the plasma to separate from the blood cells. (FIG. 6). Thus, shown in FIG. 6 is the correction for such gradient when the data for testosterone was normalized the testosterone measurement against another internal biomarker in accordance with an embodiment of the disclosure. In an embodiment, the mean coefficient of variability (CV) for the absolute measurements from six separate samplings of punches 1-5 is about 11.4%, whereas the mean CV for normalized measurements from the same samples is about 4.2%.

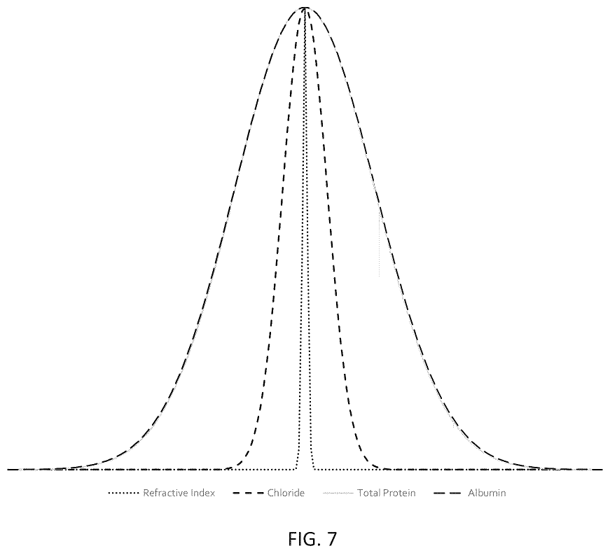

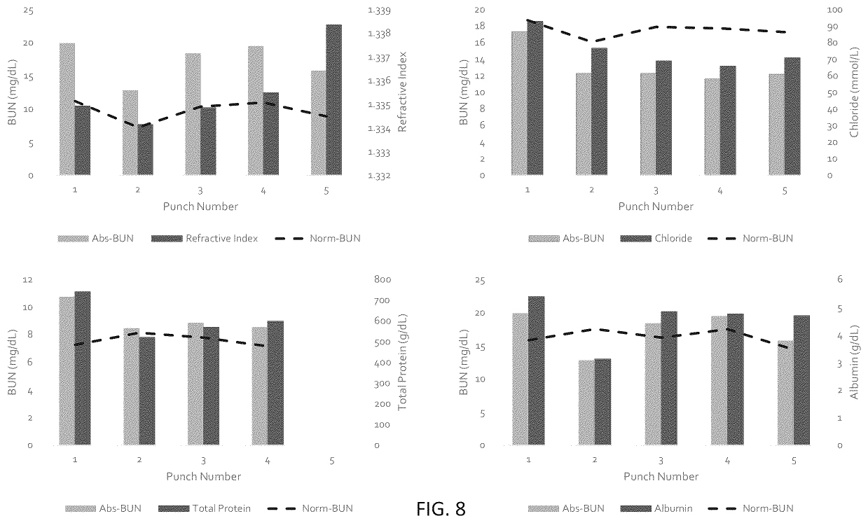

As discussed herein, the choice of a normalization marker may be based on several considerations, including a low CVg, the ease and precision of analytical measurement for the normalization marker, and whether the marker is present in the sample at a sufficient concentration. FIG. 7 shows is a graphical representation of a theoretical (based on reported CVg values) distribution of different potential markers for normalization: refractive index (CVg=0.2%) (small dashed), chloride (CVg=1.5%) (medium dash), total protein (CVg=4.7%) (solid line) and albumin (CVg=4.75%) (large dash). In an embodiment, the normalization marker with the lowest CVg and the most reliable, accurate and/or convenient method of measuring may be chosen. Thus, as shown in FIG. 8, the use of refractive index (upper left panel), chloride (upper right panel), total protein (lower left panel) and albumin (lower right panel) had varied correction for inconsistencies in sample volume and/or concentration (i.e., dilution) for BUN. In an embodiment, the normalization marker with the lowest CV (e.g., chloride in FIG. 8) may be chosen.

The samples may be used for the analysis of a biomarker by various means that are standard to the measurement of the biomarker of interest. In an embodiment, the analytical technique comprises LC-MS/MS as discussed herein.

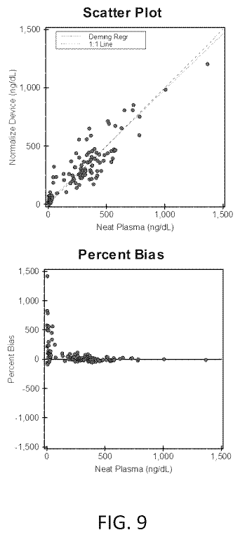

As an example, FIGS. 9-12 show the development of a LC-MS/MS assay for testosterone using dried plasma. Initial experiments employed immunoassay by an autoanalyzer using Cl ion as the normalizing marker (FIG. 9). In an embodiment, there may be significant bias at very low concentrations due at least in part to the inability to employ larger amounts of the diluted sample with the autoanalyzer.

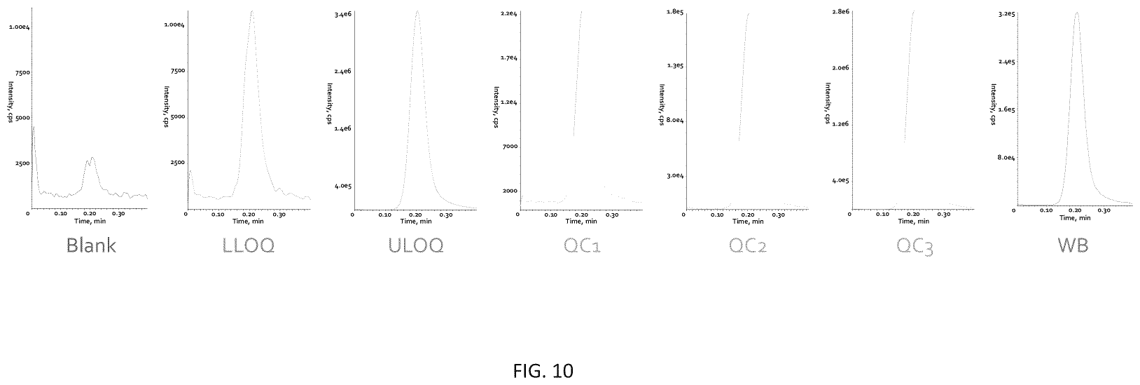

In an embodiment, the procedure employed for LC-MS/MS may comprise adding both quality controls and calibrators to the solid substrate sampling paper not containing plasma and allowed to migrate the length of the sampling paper and dry. Punches are taken from calibrators and quality controls mimicking punch position of unknown blood samples (e.g., punches 4 and 5). Next, a diluent with a stable isotope internal standard (e.g., ¹³C₄-testosterone) may be added to the punches (e.g., an approximate 12.5 fold dilution) and the extraction conducted by heating and/or shaking the punches that are immersed in the diluent. At this point the extracted samples may be pooled (i.e., punches 4 and 5 for each sample) and then an aliquot removed for analysis of the normalization marker (e.g., Cl ion) and an aliquot removed for LC-MS/MS. The aliquot used for LC-MS/MS may be partially purified by supported liquid extraction (SLE) and then an aliquot used for LC-MS/MS. Example chromatograms for testosterone at the lower limit of quantitation of 10 ng/dL (LLOQ), the upper limit of quantitation of 4945 ng/dL (ULOQ), as well as three values for the quality control samples ranging from 25 ng/dL to 3826 ng/dL, and plasma isolated from whole blood (WB) to emulate a finger stick are shown in FIG. 10. In an embodiment, the CV is ≤20% even at the lowest concentrations of the biomarker of interest for both absolute as well as normalized measurements. Also in an embodiment, normalization significantly reduces the CV and bias (FIG. 10) (Table 2).

TABLE 2

| | Absolute Measures | | | Normalized Measures | | |
|---|---|---|---|---|---|---|
| | Calibrator | QC | WB | Calibrator | QC | WB |
| Conc. (ng/dL) | 10-4945 | 25-3826 | 40-205 | 10-4975 | 26-3913 | 40-211 |
| CV (%) | <17.9 | <9.2 | <11.0 | <15.0 | <8.9 | <6.5 |
| Bias (%) | <7.1 | <3.3 | NA | <4.9 | <4.6 | NA |
| Minimum N | 24 | 24 | 15 | 24 | 24 | 15 |

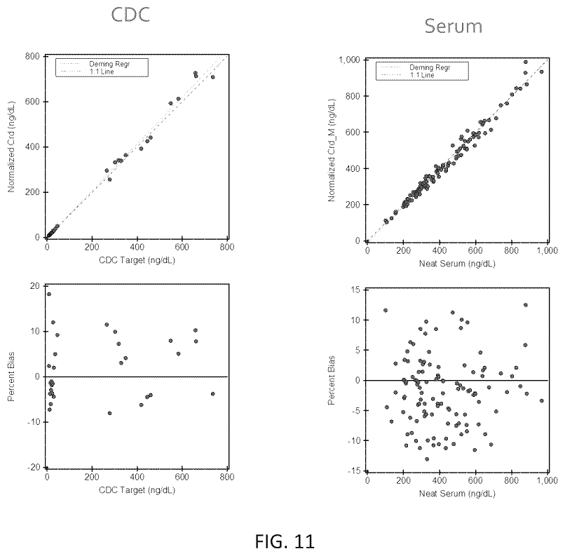

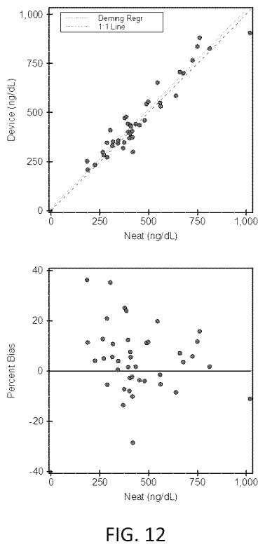

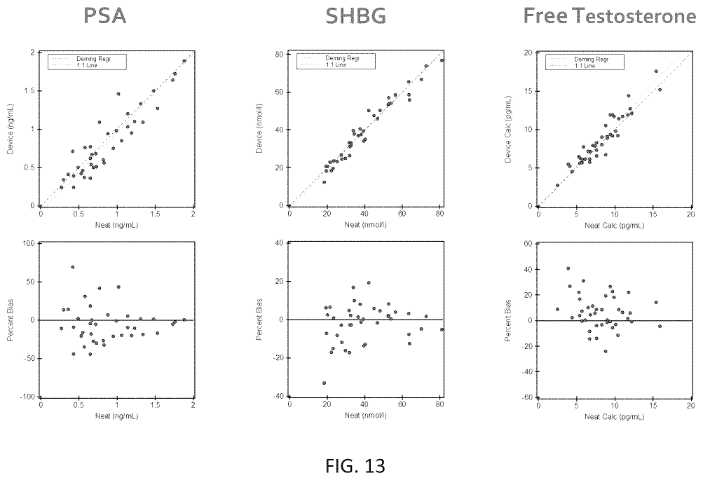

In an embodiment, the accuracy of the assay (FIG. 12) for finger stick blood samples (FIG. 12) is equivalent to CDC measured controls and spotted serum samples (FIG. 11). Also, in an embodiment, the samples may be used for the analysis of more than one biomarker. For example, in an embodiment, samples used to measure total testosterone (FIG. 12) may be used to measure additional biomarkers such as PSA and sex hormone binding globulin (SHBG) (FIG. 13). In an embodiment, the values of SHBG and testosterone for each sample may be used to determine the level of free testosterone (free T) using the Vermuelen equation (FIG. 13).

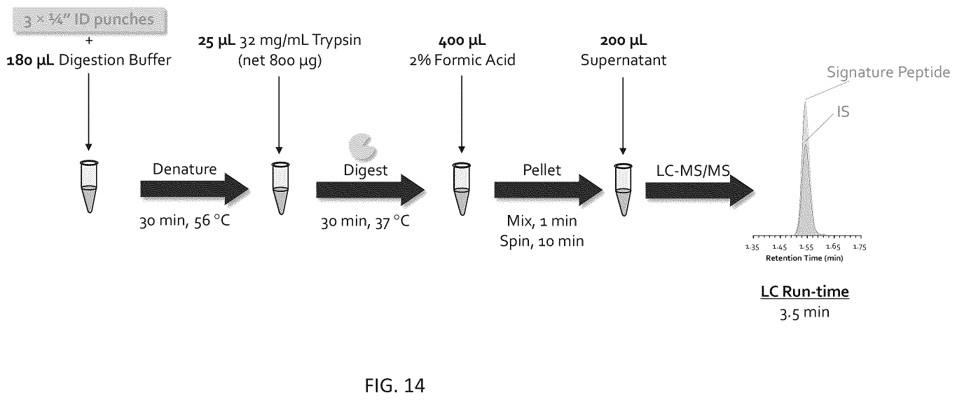

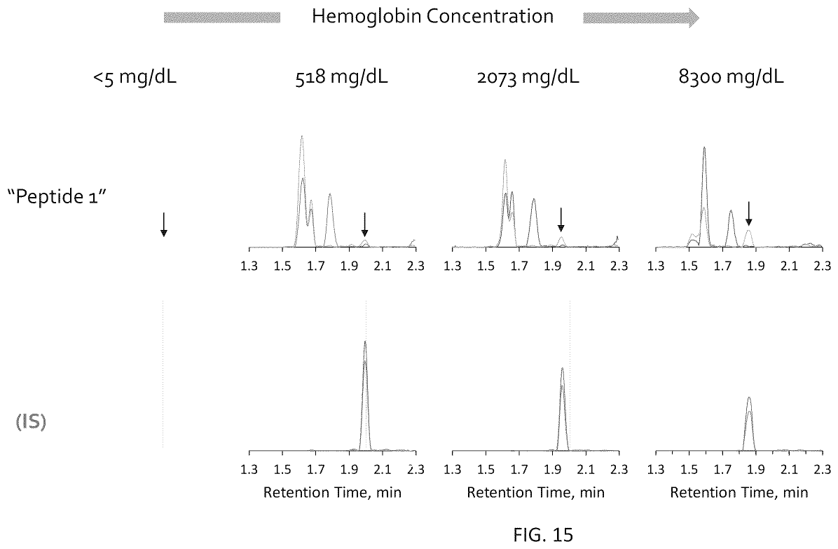

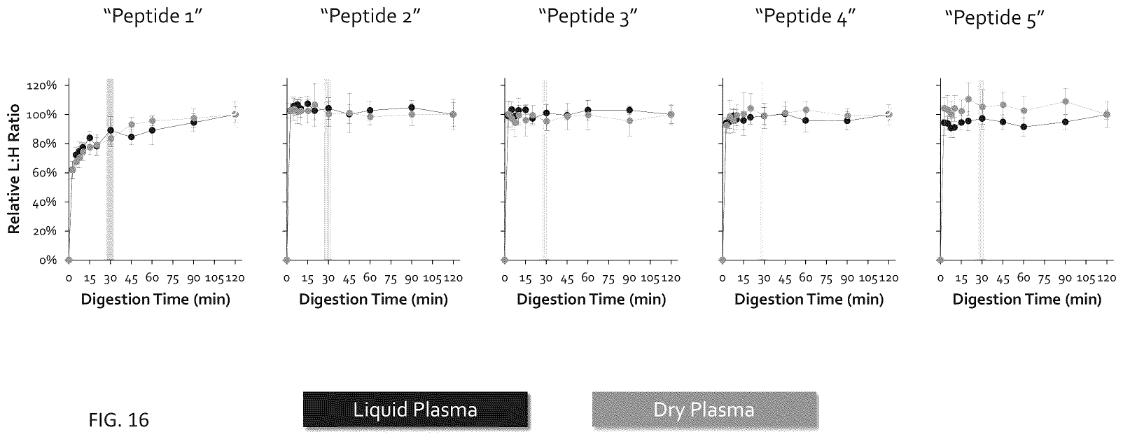

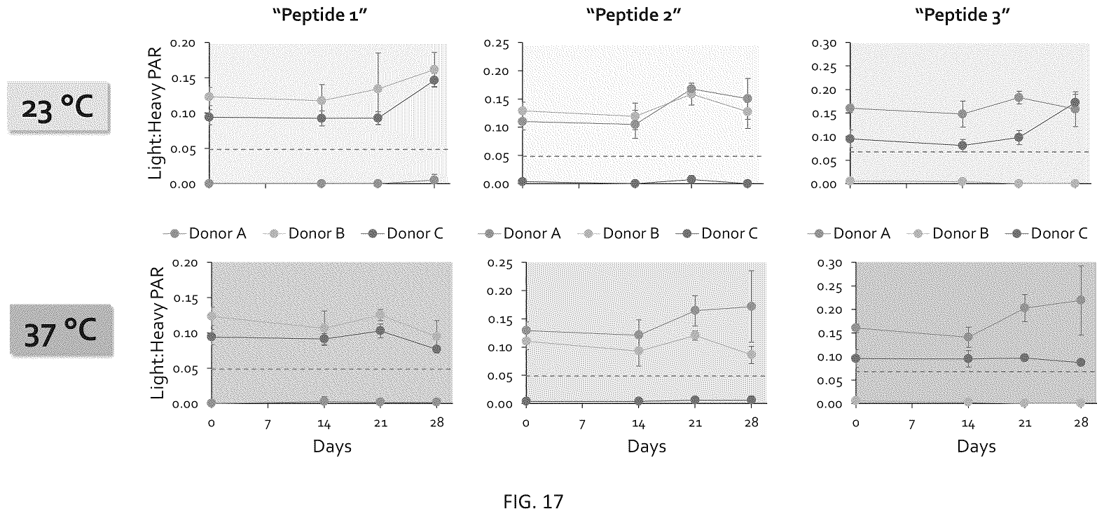

In an embodiment, the use of dried plasma may be used for the analysis of other biomarkers (e.g., nucleic acids and proteins). For example, as illustrated in FIG. 14, peptides may be generated from a protein and the measurement of the peptides may be used to evaluate the amount of the protein, or in some cases, the presence of a mutation characteristic of a disease. For example, as shown in FIG. 14, plasma punches can then be subjected to digestion (and extraction) with a protease (i.e., to generate genotype-specific peptides) and then the peptides eluted from the strip analyzed by LC-MS/MS. In an embodiment, plasma punches have less interference than blood punches (FIG. 15). The sample (i.e., sampling paper punches comprising dried plasma) may be added to digestion buffer (e.g., 0.675 mM dithiothreitol (DTT), 6.75 mg/mL deoxycholate (DOC), 50 mM Tris-HCL at pH 8.0±0.1. After a short incubation at an elevation to denature proteins present in the sample, trypsin and an internal standard may be added, and digestion is allowed to proceed to generate analyte peptides. In an embodiment, digestion is similar to that seen with plasma samples, and complete after 30 minutes (FIG. 16). Next formic acid is added to terminate the trypsin digestion and precipitate acid-insoluble materials (i.e., deoxycholate) and an aliquot of the supernatant is added to the LC-MS/MS system. In an embodiment the peptides are stable for several days both at room temperature and at an elevated temperature for LC-MS/MS (FIG. 17). In certain embodiments, the time from tube to autosampler is 90 minutes and the overall procedure only takes 260 minutes.

Systems for Simplified Biosample Processing for LC-MS/MS

Other disclosed embodiments comprise systems. For example, the disclosure herein provides a system for determining the presence and/or amount of a biomarker of interest in a biological sample comprising a body fluid, the system comprising: a device for providing a test sample comprising a dried body fluid; and a station for analyzing the by mass spectrometry to determine the presence or amount of the biomarker of interest in the biological sample. For example, in certain embodiments, the system may comprise: a station for providing a biological sample believed to contain at least one biomarker of interest wherein the biological sample comprises dried plasma; a station for extracting the at least one biomarker of interest and at least one normalizing marker from the dried plasma sample into a liquid solution; a station for measuring the at least one biomarker of interest in the liquid solution by mass spectrometry; a station for measuring the at least one normalizing marker in the liquid solution; and a station for using the ratio of the measurements for the at least one biomarker of interest and the at least one normalizing marker to determine the presence or amount of the at least one biomarker of interest in the biological sample. Or other body fluid other than plasma may be used.

In an embodiment, the mass spectrometry is operated in an atmospheric pressure chemical ionization (APCI) mode. Also in certain embodiments, at least one of the stations is automated and/or controlled by a computer. For example, as described herein, in certain embodiments, at least some of the steps are automated such that little to no manual intervention is required.

In one embodiment, the station for chromatographic separation comprises at least one apparatus to perform liquid chromatography (LC). In one embodiment, the station for liquid chromatography comprises a column for extraction chromatography. Additionally or alternatively, the station for liquid chromatography comprises a column for analytical chromatography. In certain embodiments, the column for extraction chromatography and analytical chromatography comprises a single station or single column. For example, in one embodiment, liquid chromatography is used to purify the biomarker of interest from other components in the sample that co-purify with the biomarker of interest after extraction or dilution of the sample.

The system may also include a station for analyzing the chromatographically separated one or more biomarkers of interest by mass spectrometry to determine the presence or amount of the one or more biomarkers in the test sample. In certain embodiments, tandem mass spectrometry is used (MS/MS). For example, in certain embodiments, the station for tandem mass spectrometry comprises an Applied Biosystems API4000 or API5000 or thermo quantum or Agilent 7000 triple quadrupole mass spectrometer or an Applied Biosystems API5500 or API6500 triple quadrupole or thermo Q-Exactive mass spectrometer.

The system may also comprise a station for partially from the biological sample and/or diluting the sample. In an embodiment, the station for purifying comprises a station for supported liquid extraction (SLE) and/or liquid-liquid extraction (e.g., for smaller molecules and/or lipids). For example, the station for liquid-liquid extraction may comprise equipment and reagents for addition of solvents to the sample and removal of waste fractions. Or, the station for purifying may comprise protein precipitation. Or, the station for purifying may comprise immunoaffinity enrichment (e.g., for proteins or peptides).

In some cases, an isotopically-labeled internal standard is used to standardize losses of the biomarker that may occur during the procedures. Thus, the station for SLE and/or liquid-liquid extraction may comprise a station for adding the internal standard and/or a hood or other safety features required for working with solvents.

Also, in some embodiments, the system may comprise a station for adding a quality control and/or a calibration standard. In certain embodiments, this station may be combined with the station for providing a test sample comprising a dried body fluid.

In certain embodiments, the methods and systems of the present invention may comprise multiple liquid chromatography steps. Thus, in certain embodiments, a two-dimensional liquid chromatography (LC) procedure is used. For example, in one embodiment, the method and systems of the present invention may comprise transferring the sample, or peptides derived from the sample, from a LC extraction column to an analytical column. In one embodiment, the transferring from the extraction column to an analytical column is done by a heart-cutting technique. In another embodiment, transfer from the extraction column to an analytical column by a chromatofocusing technique. Alternatively, transfer from the extraction column to an analytical column may be done by a column switching technique. These transfer steps may be done manually, or may be part of an on-line system.

Various columns comprising stationary phases and mobile phases that may be used for extraction or analytical liquid chromatography are described herein. The column used for extraction liquid chromatography may be varied depending on the biomarker of interest. In some embodiments, the extraction column is a functionalized silica or polymer-silica hybrid or polymeric particle or monolithic silica stationary phase, such as a Poroshell SBC-18 column. The column used for analytical liquid chromatography may be varied depending on the analyte and/or the column that was used for the extraction liquid chromatography step. For example, in certain embodiments, the analytical column comprises particles having an average diameter of about 5 μm. In some embodiments, the analytical column is a functionalized silica or polymer-silica hybrid, or a polymeric particle or monolithic silica stationary phase, such as a phenyl-hexyl functionalized analytical column. As noted herein, in certain embodiments, the mass spectrometer may comprise a tandem mass spectrometer (MS/MS). For example, in one embodiment of the methods and systems of the present invention, the tandem MS/MS spectrometry comprises a triple quadrupole tandem mass spectrometer. In other embodiments, the tandem mass spectrometer may be a hybrid mass spectrometer, such as a quadrupole-orbit rap or a quadrupole-time-of-flight mass spectrometer.

The tandem MS/MS may be operated in a variety of modes. In one embodiment, the tandem MS/MS spectrometer is operated in an atmospheric pressure chemical ionization (APCI) mode or Electrospray Ionization (ESI). In some embodiments, the quantification of the analytes and internal standards is performed in the selected reaction monitoring mode (SRM).

The systems and methods of the present invention may, in certain embodiments, provide for a multiplexed or high throughput assay. For example, certain embodiments may comprise a multiplexed liquid chromatography tandem mass spectrometry (LC-MS/MS) or two-dimensional or tandem liquid chromatography-tandem mass spectrometry (LC)-LC-MS/MS) methods for the proteomic analysis.

In some embodiments, a tandem MS/MS system is used. As is known by those of skill in the art, in tandem MS spectrometry, the precursor ion is selected following ionization, and that precursor ion is subjected to fragmentation to generate product (i.e., fragment) ions, whereby one or more product ions are subjected to a second stage of mass analysis for detection.

The analyte of interest may then be detected and/or quantified based upon the amount of the characteristic transitions measured by tandem MS. In some embodiments, the tandem mass spectrometer comprises a triple quadrupole mass spectrometer. In some embodiments, the tandem mass spectrometer is operated in a positive ion Atmospheric Pressure Chemical Ionization (APCI) mode. In some embodiments, the quantification of the analytes and internal standards is performed in the selected reaction monitoring mode (SRM). Or, other methods of ionization such as the use of inductively coupled plasma, or MALDI, or SELDI, ESI, or APPI may be used for ionization.

In some embodiments, the back-calculated amount of each analyte in each sample may determine by comparison of unknown sample response or response ratio when employing internal standardization to calibration curves generated by spiking a known amount of purified analyte material into a standard test sample, e.g., charcoal stripped human serum. In one embodiment, calibrators are prepared at known concentrations and analyzed as per the biomarker methodology to generate a response or response ratio when employing internal standardization versus concentration calibration curve.

The temperature for heating the sample during ionization may, in alternate embodiments range from 100° C. to about 1000° C. and includes all ranges therein. In an embodiment, the dehydration step is performed within the interface of the mass spectrometer employed in APCI or electrospray mode at 500 degrees C.±100 degrees. In an embodiment, the sample is heated for several microseconds at the interface for dehydration to occur. In alternate embodiments, the heating step is done for less than 1 second, or less than 100 milliseconds (msec), or less than 10 msec, or less than 1 msec, or less than 0.1 msec, or less than 0.01 msec, or less than 0.001 msec.

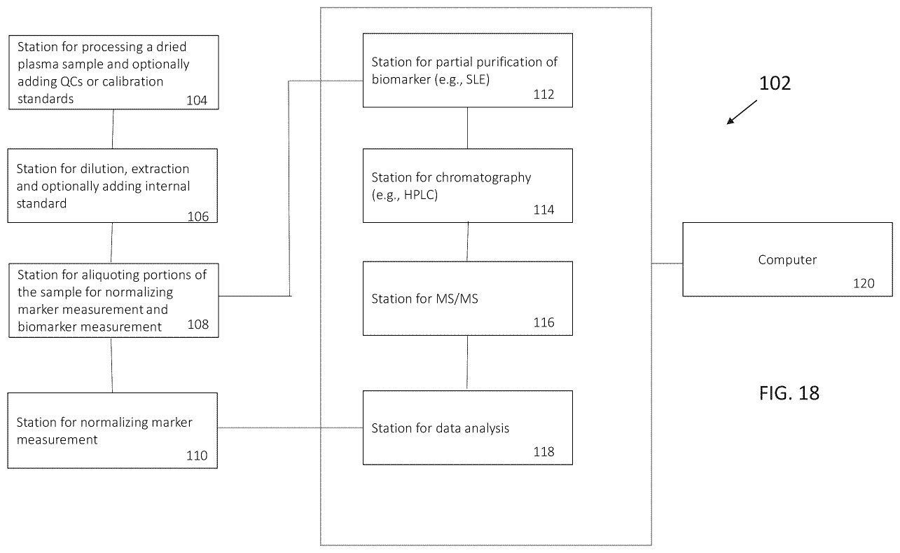

FIG. 18 shows an embodiment of a system (102) of the present invention. For the systems described herein, there may be additional stations and/or some of the stations may be combined. For example, as shown in FIG. 18, the system may comprise a station for processing a sample comprising dried plasma (e.g., immobilized on a substrate) (104) that may comprise a biomarker of interest into sampling containers (e.g., 96 well microtiter assay wells). The station for processing the sample may include components for adding at least one of a quantity control (QC) and/or calibration standard to the dried plasma sample. In one embodiment, the sample is aliquoted into a container or containers to facilitate extraction at an extraction station (106) of the biomarker of interest. The station for aliquoting may comprise receptacles to discard the portion of the biological sample that is not used in the analysis.

The extraction station (106) may further comprise a station for adding an internal standard to the sample. In an embodiment, the internal standard comprises the biomarker of interest labeled with a non-natural isotope. Thus, the station for extraction and/or adding an internal standard may comprise safety features to facilitate adding an isotopically labeled internal standard solutions to the sample.

Where LC-MS/MS is not used to measure the normalizing marker, the system may comprise a station for aliquoting portions of the extracted sample (108) for measurement of a normalization marker and measurement of the biomarker of interest. The system may also comprise a station (110) for measuring the normalization marker with a technique that is independent of mass spectrometry. The technique used will depend on the nature of the normalization (normalizing) standard, but may be one of any of the techniques known to one of skill in the art.

The system may also comprise a station for further processing of the biomarker. For example, the system may also, in some embodiments, comprise a station (112) for purification steps such as supported liquid extraction, liquid-liquid extraction, protein precipitation and/or dilution of the sample.

The system may also comprise a station for liquid chromatography (e.g., HPLC) of the sample (114). As described herein, in an embodiment, the station for liquid chromatography may comprise an extraction liquid chromatography column. The station for liquid chromatography may comprise a column comprising the stationary phase, as well as containers or receptacles comprising solvents that are used as the mobile phase. In an embodiment, the mobile phase comprises a gradient of methanol and water, acetonitrile and water, or other miscible solvents with aqueous volatile buffer solutions. Thus, in one embodiment, the station may comprise the appropriate lines and valves to adjust the amounts of individual solvents being applied to the column or columns. Also, the station may comprise a means to remove and discard those fractions from the LC that do not comprise the biomarker of interest. In an embodiment, the fractions that do not contain the biomarker of interest are continuously removed from the column and sent to a waste receptacle for decontamination and to be discarded. The system may also comprise an analytical LC column (114). The analytical column may facilitate further purification and concentration of the biomarker of interest as may be required for further characterization and quantification.

Also, the system may comprise a station for characterization and quantification of the biomarker of interest. In one embodiment, the system may comprise a station for mass spectrometry (MS) (116) of the biomarker. In an embodiment, the station for mass spectrometry comprises a station for tandem mass spectrometry (MS/MS). Also, the station for characterization and quantification may comprise a station for data analysis (118) for the biomarker and the normalization marker and/or a computer (120) and software for analysis of the results. In an embodiment, the analysis comprises both identification and quantification of the biomarker of interest.

In some embodiments, one or more of the purification or separation steps can be performed “on-line.” As used herein, the term “on-line” refers to purification or separation steps that are performed in such a way that the test sample is disposed, e.g., injected, into a system in which the various components of the system are operationally connected and, in some embodiments, in fluid communication with one another. The on-line system may comprise an autosampler for removing aliquots of the sample from one container and transferring such aliquots into another container. For example, an autosampler may be used to transfer the sample after extraction onto an LC extraction column. Additionally or alternatively, the on-line system may comprise one or more injection ports for injecting the fractions isolated from the LC extraction columns onto the LC analytical column. Additionally or alternatively, the on-line system may comprise one or more injection ports for injecting the LC purified sample into the MS system. Thus, the on-line system may comprise one or more columns, including but not limited to, an extraction column, including an HTLC extraction column, and in some embodiments, an analytical column. Additionally or alternatively, the system may comprise a detection system, e.g., a mass spectrometer system. The on-line system may also comprise one or more pumps; one or more valves; and necessary plumbing. In such “on-line” systems, the test sample and/or analytes of interest can be passed from one component of the system to another without exiting the system, e.g., without having to be collected and then disposed into another component of the system.

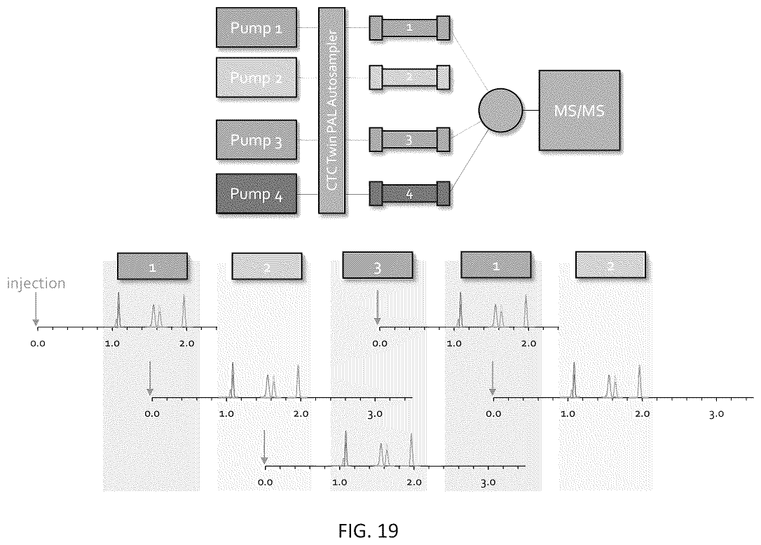

In some embodiments, the on-line purification or separation method can be automated. In such embodiments, the steps can be performed without the need for operator intervention once the process is set-up and initiated. Thus, in various embodiments, the system, or portions of the system may be controlled by a computer or computers (120). Thus, in certain embodiments, the present invention may comprise software for controlling the various components of the system, including pumps, valves, autosamplers, and the like. Such software can be used to optimize the extraction process through the precise timing of sample and solute additions and flow rate. For example, as shown in FIG. 19, when employed on a multiplexing LC system, such as the ARI Transcent™ TLX-4, injections may be run in parallel with injections staggered every 1.5 minutes to improve the duty cycle of the mass spectrometric analysis.

Although some or all of the steps in the method and the stations comprising the system may be on-line, in certain embodiments, some or all of the steps may be performed “off-line.” In contrast to the term “on-line”, the term “off-line” refers to a purification, separation, or extraction procedure that is performed separately from previous and/or subsequent purification or separation steps and/or analysis steps. In such off-line procedures, the analytes of interest typically are separated, for example, on an extraction column or by liquid/liquid extraction, from the other components in the sample matrix and then collected for subsequent introduction into another chromatographic or detector system. Off-line procedures typically require manual intervention on the part of the operator.

Liquid chromatography may, in certain embodiments, comprise high turbulence liquid chromatography or high throughput liquid chromatography (HTLC). See, e.g., Zimmer et al., J. Chromatogr. A 854:23-35 (1999); see also, U.S. Pat. Nos. 5,968,367; 5,919,368; 5,795,469; and 5,772,874. Traditional HPLC analysis relies on column packings in which laminar flow of the sample through the column is the basis for separation of the analyte of interest from the sample. In such columns, separation is a diffusional process. Turbulent flow, such as that provided by HTLC columns and methods, may enhance the rate of mass transfer, improving the separation characteristics provided. In some embodiments, high turbulence liquid chromatography (HTLC), alone or in combination with one or more purification methods, may be used to purify the biomarker of interest prior to mass spectrometry. In such embodiments, samples may be extracted using an HTLC extraction cartridge which captures the analyte, then eluted and chromatographed on a second HTLC column or onto an analytical HPLC column prior to ionization. Because the steps involved in these chromatography procedures can be linked in an automated fashion, the requirement for operator involvement during the purification of the analyte can be minimized. Also, in some embodiments, the use of a high turbulence liquid chromatography sample preparation method can eliminate the need for other sample preparation methods including liquid-liquid extraction. Thus, in some embodiments, the test sample, e.g., a biological fluid, can be disposed, e.g., injected, directly onto a high turbulence liquid chromatography system.

For example, in a typical high turbulence or turbulent liquid chromatography system, the sample may be injected directly onto a narrow (e.g., 0.5 mm to 2 mm internal diameter by 20 to 50 mm long) column packed with large (e.g., >25 micron) particles. When a flow rate (e.g., 3-500 mL per minute) is applied to the column, the relatively narrow width of the column causes an increase in the velocity of the mobile phase. The large particles present in the column can prevent the increased velocity from causing back pressure and promote the formation of vacillating eddies between the particles, thereby creating turbulence within the column.

In high turbulence liquid chromatography, the analyte molecules may bind quickly to the particles and typically do not spread out, or diffuse, along the length of the column. This lessened longitudinal diffusion typically provides better, and more rapid, separation of the analytes of interest from the sample matrix. Further, the turbulence within the column reduces the friction on molecules that typically occurs as they travel past the particles. For example, in traditional HPLC, the molecules traveling closest to the particle move along the column more slowly than those flowing through the center of the path between the particles. This difference in flow rate causes the analyte molecules to spread out along the length of the column. When turbulence is introduced into a column, the friction on the molecules from the particle is negligible, reducing longitudinal diffusion.

The methods and systems of the present invention may use mass spectrometry to detect and quantify the biomarker of interest. The terms “mass spectrometry” or “MS” as used herein generally refer to methods of filtering, detecting, and measuring ions based on their mass-to-charge ratio, or “m/z.” In MS techniques, one or more molecules of interest are ionized, and the ions are subsequently introduced into a mass spectrometer where, due to a combination of electric fields, the ions follow a path in space that is dependent upon mass (“m”) and charge (“z”).

In certain embodiments, the mass spectrometer uses a “quadrupole” system. In a “quadrupole” or “quadrupole ion trap” mass spectrometer, ions in an oscillating radio frequency (RF) field experience a force proportional to the direct current (DC) potential applied between electrodes, the amplitude of the RF signal, and m/z. The voltage and amplitude can be selected so that only ions having a particular m/z travel the length of the quadrupole, while all other ions are deflected. Thus, quadrupole instruments can act as both a “mass filter” and as a “mass detector” for the ions injected into the instrument.

In certain embodiments, tandem mass spectrometry is used. See, e.g., U.S. Pat. No. 6,107,623, entitled “Methods and Apparatus for Tandem Mass Spectrometry,” which is hereby incorporated by reference in its entirety. Further, the selectivity of the MS technique can be enhanced by using “tandem mass spectrometry,” or “MS/MS.” Tandem mass spectrometry (MS/MS) is the name given to a group of mass spectrometric methods wherein “parent or precursor” ions generated from a sample are fragmented to yield one or more “fragment or product” ions, which are subsequently mass analyzed by a second MS procedure. MS/MS methods are useful for the analysis of complex mixtures, especially biological samples, in part because the selectivity of MS/MS can minimize the need for extensive sample clean-up prior to analysis. In an example of an MS/MS method, precursor ions are generated from a sample and passed through a first mass filter to select those ions having a particular mass-to-charge ratio. These ions are then fragmented, typically by collisions with neutral gas molecules in a suitable ion containment device, to yield product (fragment) ions, the mass spectrum of which is recorded by an electron multiplier detector. The product ion spectra so produced are indicative of the structure of the precursor ion, and the two stages of mass filtering can eliminate ions from interfering species present in the conventional mass spectrum of a complex mixture.

In an embodiment, the methods and systems of the present invention use a triple quadrupole MS/MS (see e.g., Yost, Enke in Ch. 8 of Tandem Mass Spectrometry, Ed. McLafferty, pub. John Wiley and Sons, 1983). Triple quadrupole MS/MS instruments typically consist of two quadrupole mass filters separated by a fragmentation means. In one embodiment, the instrument may comprise a quadrupole mass filter operated in the RF only mode as an ion containment or transmission device. In an embodiment, the quadrupole may further comprise a collision gas at a pressure of between 1 and 10 millitorr. Many other types of “hybrid” tandem mass spectrometers are also known, and can be used in the methods and systems of the present invention including various combinations of orbitrap analyzers and quadrupole filters. These hybrid instruments often comprise high resolution orbitrap analyzers (see e.g., Hu Q, Noll R J, Li H, Makarov A, Hardman M, Graham Cooks R. The Orbitrap: a new mass spectrometer. J Mass Spectrom. 2005; 40(4):430-443) for the second stage of mass analysis. Use of high resolution mass analyzer may be highly effective in reducing chemical noise to very low levels.

For the methods and systems of the present invention, ions can be produced using a variety of methods including, but not limited to, electron ionization, chemical ionization, fast atom bombardment, field desorption, and matrix-assisted laser desorption ionization (“MALDI”), surface enhanced laser desorption ionization (“SELDI”), photon ionization, electrospray ionization, and inductively coupled plasma.

In those embodiments, such as MS/MS, where precursor ions are isolated for further fragmentation, collision-induced dissociation (“CID”) may be used to generate the fragment ions for further detection. In CID, precursor ions gain energy through collisions with an inert gas, and subsequently fragment by a process referred to as “unimolecular decomposition.” Sufficient energy must be deposited in the precursor ion so that certain bonds within the ion can be broken due to increased vibrational energy.

In some embodiments, to attain the required analytical selectivity and sensitivity, the presently disclosed 2D-LC-MS/MS methods include multiplexed sample preparation procedures. For example, in certain embodiments dialysis of the sample is performed using a 96 well plate having a dialysis membrane in each well or multiple sample tubes. Additionally or alternatively, the multiplex system may comprise staggered multiplexed LC and MS sample inlet systems. Also, the methods and systems of the present invention may comprise multiple column switching protocols, and/or heart-cutting (LC-LC or 2D-LC) techniques, and/or LC separations prior to MS detection. In some embodiments, the methods and systems of the present invention may include a multiplexed two-dimensional liquid chromatographic system coupled with a tandem mass spectrometer (MS/MS) system, for example a triple quadrupole MS/MS system. Such embodiments provide for staggered, parallel sample input into the MS system.

Thus, multiple samples may each be applied to individual extraction columns. Once the samples have each run through the extraction column, they may each be transferred directly (e.g., by column switching) to a second set of analytical columns. As each sample elutes from the analytical column, it may be transferred to the mass spectrometer for identification and quantification.

A plurality of analytes can be analyzed simultaneously or sequentially by the presently disclosed LC-MS/MS and 2D-LC-MS/MS methods. Exemplary analytes amenable to analysis by the presently disclosed methods include, but are not limited to, peptides, steroid hormones, nucleic acids, vitamins and the like. One of ordinary skill in the art would recognize after a review of the presently disclosed subject matter that other similar analytes could be analyzed by the methods and systems disclosed herein. Thus, in alternate embodiments, the methods and systems may be used to quantify steroid hormones, protein and peptide hormones, peptide and protein biomarkers, drugs of abuse and therapeutic drugs. For example, optimization of key parameters for each analyte can be performed using a modular method development strategy to provide highly tuned bioanalytical assays. Thus, certain steps may be varied depending upon the analyte being measured as disclosed herein.

Also, embodiments of the methods and systems of the present invention may provide equivalent sensitivity attainable for many of the analytes being measured using much less sample. For example, through using this optimization procedure, an LLOQ of about 10 nmole/L for detection of a peptide biomarker for dried plasma corresponding to about 20 μL of liquid plasma. Such small sample sizes render sampling (often by finger-prick) much more accessible.

### EXAMPLES

### Example 1

The concentration of testosterone in blood plasma was determined from deposition of blood onto a laminar flow paper (i.e., a sampling paper), allowing for separation of the plasma fraction and red blood cells which are subsequently dried. The paper was stored in a sealed pouch containing desiccant and oxygen scrubber to preserve the integrity of the dry plasma specimen. Subsequently, testosterone (i.e., the biomarker) was measured in extracted dry plasma and the blood plasma concentration determined through normalization with a measurement of chloride ion in the same extracted dry plasma specimen.

Following spotting of blood and separation of red cells and plasma on the laminar flow sampling paper, the paper was allowed to dry for a period ≥60 minutes. Punches of dry plasma (⅜ inch square, approximating 20 μL of liquid plasma) from the laminar flow sampling paper were used to extract the test specimen in a 16×75 mm tube for 2.5 hours on an orbital mixer using 250 μL of 0.1% BSA solution containing stable isotope labeled testosterone. Extracts were split into matching aliquots—one aliquot for quantification of testosterone by LC-MS/MS after further purification by supported liquid extraction and one aliquot for quantification of chloride ion in the extract using colorimetric determination.

Examination of the dried blood collection and specimen reconstitution process could lead to concerns that specimen recovery rates may vary. The use of a reference analyte or normalizing marker, internal to the specimen that is present throughout the collection, punching, reconstitution, and extraction processes, permits the assessment of the efficiency of the complete process. Chloride is relatively consistent in the healthy population (inter-individual coefficient of variation=1.5%) making it a logical selection as this internal reference (Ricos C. et al., Scand J Clin Lab Invest 1999; 59:491-500). Normalization of the biomarker measurement in extracted dry plasma, thus, may be based upon the measurement of the normalizing chloride marker measurement in extracted dry plasma as compared to its expected value in blood plasma, such as a defined population average chloride value of 100.28 millimoles per liter (mM) measured in liquid blood plasma/serum. Serum albumin may be utilized as an alternative reference analyte to chloride because of its low subject-to-subject variation (inter-individual coefficient of variation=4.8%) and for this laboratory has a population average value of 4.31 grams per deciliter.

The premise of this is based on the understanding that the chloride concentration in the blood plasma deposited onto the strip should match a known value, the population average for chloride, with a low degree of uncertainty, such that measurement of the chloride ion in the extracted dry plasma will allow establishment of the recovery of the chloride ion measurement. For example, if a measurement of 68 mM is obtained for chloride in the extracted dry plasma, this indicates a recovery of 67.8% relative to the expected blood plasma concentration (67.8%=68 mM/100.28 mM×100). As such, it may be established that the same recovery would occur to the testosterone, such that the testosterone measurement in the dry plasma extract may be normalized to the chloride recovery to determine the concentration of testosterone in the blood plasma originally deposited onto the laminar flow sampling paper. This is accomplished by dividing the testosterone measurement in the extracted dry plasma specimen by the chloride measurement in the extracted dry plasma specimen, which is multiplied by the expected concentration of chloride in the blood plasma.

Given the chemical properties of the biomarker (testosterone) and normalizing marker (chloride) are different, it may not be necessary that their recovery through the processes of collection, reconstitution, and extraction, which would preclude accurate determination of the testosterone blood plasma concentrations as described above. Thus, it may be necessary to establish their relative recovery, which should be reproducible with proper optimization of extraction procedures. In this manner, the chloride-normalized testosterone measurement as described above would be multiplied by a correction factor which is the expected relative recovery of chloride and testosterone in the extracted dry plasma. For example, if the testosterone measurement in the extracted dry plasma recovers 10% of the blood plasma concentration on average and the chloride measurement in the extracted dry plasma recovers 68% of the blood plasma chloride concentration on average, then the relative recovery of chloride and testosterone in extracted dry plasma would be 6.8 (68%/10%). Thus, for example a testosterone measurement of 50 ng/dL is the extracted dry plasma and a chloride measurement of 67.8 nM, the concentration of testosterone in blood plasma is estimated as 50 ng/dL/67.8 nM×100.28 nM×6.8 or 502.9 ng/dL.

In some instances, it may be sufficient to simply derive and report a concentration of testosterone in the dry plasma, rather than determine the blood plasma concentration from a dry plasma sample. In such cases, it will be advantageous to report the testosterone concentration in dry plasma in units of amount of testosterone per unit of normalizing chloride (e.g., nanograms of testosterone per millimole of chloride) due to volume/extraction variance of the dry plasma measurement—analogous to normalization of urine measurements to creatinine.

### Example 2

The concentration of C-reactive protein (CRP) in blood plasma is determined from deposition of blood onto a laminar flow sampling paper, allowing for separation of the plasma fraction and red blood cells which are subsequently dried. The paper is stored in a sealed pouch containing desiccant and oxygen scrubber to preserve the integrity of the dry plasma specimen. Subsequently, CRP (i.e., the biomarker) is measured in extracted dry plasma and the blood plasma concentration determined through normalization with a measurement of albumin in the same extracted dry plasma specimen.

Following spotting of blood and separation of red cells and plasma on the laminar flow sampling paper, the paper is allowed to dry for a period ≥60 minutes. Punches of dry plasma (3×¼″ ID round, approximating 20 μL of liquid plasma) from the laminar flow sampling paper are used to extract and denature the test specimen in a microcentrifuge tube for 0.5 hours at 56° C. using 180 μL of denaturing buffer (0.675 mM dithiothreitol, 0.675 mg/mL sodium deoxycholate, 50 mM tris-HCl, pH 8.0). Subsequently, stable isotope labeled internal standards are added to the denatured extracts, which are digested with 800 ug of bovine trypsin for 0.5 hours at 37° C. after to produce proteolytic surrogate peptides specific to CRP and serum albumin, having the amino acid sequences of ESDTSYVSLK (SEQ ID NO. 1) and AEFAEVSK (SEQ ID NO. 2), respectively. Subsequently, both surrogate peptides and their respective internal standards are measured by LC-MS/MS to determine the quantity of CRP and albumin in the extracted dry plasma.

Examination of the dried blood collection and specimen reconstitution process could lead to concerns that specimen recovery rates may vary. The use of a reference analyte or normalizing marker, internal to the specimen that is present throughout the collection, punching, reconstitution, and extraction processes, permits the assessment of the efficiency of the complete process. Serum albumin is relatively consistent in the healthy population (CVg=4.8%) making it a logical selection as this internal reference (Ricos C. et al., Scand J Clin Lab Invest 1999; 59:491-500), particularly for other protein biomarkers. Normalization of the biomarker measurement in extracted dry plasma, thus, may be based upon the measurement of the normalizing albumin marker measurement in extracted dry plasma as compared to its expected value in blood plasma, such as this laboratory's population average albumin value of 4.31 grams per deciliter (g/dL) measured in liquid blood plasma/serum.

The premise of this is based on the understanding that the albumin concentration in the blood plasma deposited onto the strip should match a known value, the population average for albumin, with a low degree of uncertainty, such that measurement of the albumin in the extracted dry plasma will allow one to establish the recovery of the albumin measurement. For example, if a measurement of 2.7 g/dL is obtained for albumin in the extracted dry plasma, this indicates a recovery of 62.6% relative to the expected blood plasma concentration (62.6%=2.7 g/dL/4.31 g/dL×100). As such, it may be established that the same recovery would occur to the CRP, such that the CRP measurement in the dry plasma extract may be normalized to the albumin recovery to determine the concentration of CRP in the blood plasma originally deposited onto the laminar flow sampling paper. This is accomplished by dividing the CRP measurement in the extracted dry plasma specimen by the albumin measurement in the extracted dry plasma specimen, which is multiplied by the expected concentration of albumin in the blood plasma.

Given the biochemical properties and structure of the biomarker (CRP) and normalizing marker (albumin) are different, it may not be necessary that their recovery through the processes of collection, reconstitution, and extraction, which would preclude accurate determination of the CRP blood plasma concentrations as described above. Thus, it may be necessary to establish their relative recovery, which should be reproducible with proper optimization of extraction procedures. In this manner, the albumin-normalized CRP measurement as described above would be multiplied by a correction factor which is the expected relative recovery of albumin and CRP in the extracted dry plasma. For example, if the CRP measurement in the extracted dry plasma recovers 50% of the blood plasma concentration on average and the albumin measurement in the extracted dry plasma recovers 68% of the blood plasma albumin concentration on average, then the relative recovery of albumin and CRP in extracted dry plasma would be 1.36 (68%/50%). Thus, for example a CRP measurement of 3.2 mg/L is the extracted dry plasma and an albumin measurement of 2.9 g/dL, the concentration of CRP in blood plasma is estimated as 3.2 mg/L/2.9 g/L×4.31 g/L×1.36 or 6.5 mg/L of CRP.

In some instances, it may be sufficient to simply derive and report a concentration of CRP in the dry plasma, rather than determine the blood plasma concentration from a dry plasma sample. In such cases, it will be advantageous to report the CRP concentration in dry plasma in units of amount of CRP per unit of normalizing albumin (e.g., milligrams of CRP per gram of albumin) due to volume/extraction variance of the dry plasma measurement—analogous to normalization of urine measurements to creatinine.

### Example 3

Experiments were performed to characterize the methods and systems disclosed herein. FIG. 2 shows results for 20 μL serial collections from finger stick capillary blood that was collected incrementally up to 180 μL. Each 20 μL collection was diluted (12.5 fold) and analyzed on autoanalyzer for standard components of a complete blood count (CBC). It was found that measurement of each of the cellular components, white blood cells (WBC), red blood cells (RBC) and hematocrit (HCT) was consistent over the sampling volume and time.

FIG. 3 shows a similar evaluation of sampling for the biomarkers: blood urea nitrogen (BUN), glucose, cholesterol, C-reactive protein (CRP), alanine amino transferase (ALT) and albumin. In this experiment, 20 μL aliquots up to 200 μL were collected and analyzed. The first droplet after finger stick was not wiped away. Each aliquot was diluted by about 12.5-fold gravimetrically. The samples were centrifuged and the supernatant was removed and measured for each of the biomarkers. It can be seen that there is very little variability observed in biomarker distribution between 20 uL fractions. Samples were run on the development channel of commercial autoanalyzer utilizing FDA approved reagents and calibration systems. An over-aspirated sample volume was injected into the system to account for dilution and the use of small 2-3 uL of neat sample (i.e., serum or plasma).

It was found that measurements using diluted plasma from various sampling methods (venipuncture, finger stick, or transdermal) were highly correlated. Venipuncture (r=0.997) and finger stick (r=0.979) were found to fall within a mean bias of <4.1% which is a desirable mean and transdermal collection (r=0.967) was within a mean bias of <6.2%, which satisfies a minimal mean bias (see e.g., Ricos C. et al., Scand J Clin Lab Invest 1999; 59:491-500).

To assess whether there is a negative bias introduced by dilution of plasma during the procedure, a comparison of the three collection methods—diluted venipuncture (vein-diluted), diluted finger stick plasma, and diluted transdermal plasma as compared to the analysis of biomarkers from undiluted plasma from venipuncture—was performed. Biomarkers assessed were cholesterol, C-reactive protein (CRP), glucose, HDL, LDL, prostate specific antigen (PSA), triglycerides, and thyroid stimulating hormone (TSH). It was found that the mean of mean biases for diluted vein was −1.2%, for finger stick was −1.3% and for transdermal was −1.8% (FIG. 4) indicating that there was a high degree of accuracy, and a heteroscedastic distribution across 0% bias, implying a lack of interstitial fluid dilution effects.

A comparison of the level of interference for dried blood samples as compared to dried plasma is shown in FIG. 5. It can be seen (upper portion of FIG. 5) that as the sample moves from punch 1 to punch 5 plasma separates from the red blood cells (seen as the darker dried sample). When each punch was extracted and measured for testosterone it was found that the result is closer to clinical values at punches 4 and 5 than for the earlier punches.

Based on the results shown in FIG. 5, for punches 4 and 5 were used for sample extraction for patient analysis. Each punch was extracted separately and the supernatant combined and mixed prior to analysis.

Normalization markers may be used to correct for sampling inconsistences due to for example, a larger or smaller blood sample being applied to the sampling paper and/or the dilution of the biomarker(s) as the sample migrates (e.g., from punch 1 to punch 5) across the sampling device to allow the plasma to separate from the blood cells. FIG. 6 shows the correction for dilution effects when the data for testosterone was normalized to another internal biomarker. It can be seen that use of the normalization marker reduced variance due to sample migration and/or interference (e.g., hemoglobin). It was found that the mean coefficient of variability (CV) for the absolute measurements from six separate samplings of punches 1-5 (i.e., punches 1-5 were combined) was 11.4%, whereas the mean CV for normalized measurements from the same samples was 4.2%.

Shown in FIG. 7 is a graphical representation of a distribution of different potential measures for normalization. Generally, the preferred measure would be expected to be the normalizer that has the least variability between individuals in a group. It can be seen that, in this example, refractive index (CVg=0.2%) provides the least variability, followed by chloride (CVg=1.5%), followed by total protein (CVg=4.7%) and albumin (CVg=4.75%). Other suitable normalization markers are shown in Table 1. Each of these normalization markers was tested for its ability to normalize the measurement of the biomarker blood urea nitrogen (BUN) (FIG. 8). The line which is overlaid on the bar graphs is the normalized result for BUN. The normalizer with the lowest CV across the 5 punches (in this case chloride ion) was chosen as the normalizer to move forward with.

In another experiment, the ability of a normalization marker to correct for partial plasma saturation of a sampling location (i.e., a punch position) was evaluated. In this experiment, samples from two saturated punches (i.e., about 100% matrix) were combined and compared to samples from one punch that was completely saturated and one punch that was about 50% saturated (i.e., about 75% sample matrix). The % bias plot for the 75% matrix samples exhibited under recovery as expected (−25.2% bias). However, use of the normalizing marker (chloride ion) corrected the bias to −1.9%.

### Example 4

The use of dried plasma samples was applied to testing for testosterone by LC-MS/MS. (FIGS. 9-12). Initial experiments employed immunoassay by an autoanalyzer using Cl ion as the normalizing marker (FIG. 9). For the upper (scatter) plot the slope (Deming)=0.959; the correlation coefficient (R)=0.9039; and the bias was 4.079%. It can be seen, however, that there was significant bias at very low concentrations. It is expected that this bias is due at least in part to the inability to over-aspirate sample volume on the autoanalyzer immunoassay to account for the dilution imparted by extraction.

LC-MS/MS analysis of a finger stick collected sample employed the steps of applying a finger stick blood sample to a solid support (e.g., sampling paper) and allowing the plasma to separate and dry. Additionally adding both calibrators and quality controls (QC) directly to the sampling paper and allowed to migrate and dry. Punches designated for sampling (i.e., spots 4 and 5 as shown in FIG. 5).

Punches of dry plasma (⅜ inch square, approximating 20 μL of liquid plasma) from the sampling paper were used to extract the test specimen in a 16×75 mm tube for 2.5 hours on an orbital mixer using 250 μL of 0.1% BSA solution containing stable isotope labeled testosterone. Extracts were split into matching aliquots—one aliquot for quantification of testosterone by LC-MS/MS after further purification by supported liquid extraction and one aliquot for quantification of chloride ion in the extract using colorimetric determination.

Example chromatograms for testosterone at the lower limit of quantitation of 10 ng/dL (LLOQ), the upper limit of quantitation of 4945 ng/dL (ULOQ), as well as three values for the quality control samples ranging from 25 ng/dL to 3,826 ng/dL, and plasma isolated from whole blood (WB) to emulate a finger stick are shown in FIG. 10. It was found that the CV is ≤20% even at the lowest concentrations of the biomarker of interest for both absolute as well as normalized measurements, and that normalization significantly reduces the CV and bias (Table 2). Using this workflow, the labeled isotope provides for normalization for SLE and LC-MS/MS whereas the Cl⁻ provides for normalization of migration/dose volume and extraction efficiency.

The accuracy of the assay (FIG. 12) for finger stick blood samples (FIG. 12) was found to be substantially equivalent to CDC measured controls and spotted serum samples having testosterone in the >100 ng/dL range (FIG. 11). In FIG. 11, the left scatter (slope (Deming)=1.04; R=0.9966; bias=2.83%; N=30) and bias plots show spotted and extracted CDC Phase 1 Hormone Standardization samples (value assigned by the CDC). The right scatter (slope (Deming)=1.01; R=0.9904; bias=−1.649%; N=115) and bias plots are for a larger number of serum samples. This data was obtained by spotting serum having unknown levels of testosterone, that were expected to be in the >100 ng/dL range on the sampling paper, extracting, and comparing the obtained values to neat serum measurement. This assay was performed over 5 days, further confirming the accuracy in measurement and lack of calibration drift from day to day. For FIG. 12 the slope (Deming)=1.013; R=0.9567; bias=3.65%; N=41.

FIG. 13 shows correlative data obtained using other biomarkers. In this experiment, samples used to measure total testosterone (FIG. 12) were used to measure additional biomarkers such as PSA (slope (Deming)=1.03; R=0.923; bias=−5.87%; N=38) and sex hormone binding globulin (SHBG) (slope (Deming)=1.02; R=0.978; bias=−1.40%; N=38) (FIG. 13). The values of SHBG and testosterone for each sample were used to determine the level of free testosterone (free T) (slope (Deming)=1.08; R=0.943; bias=5.05%; N=41) using the Vermuelen equation.

### Example 5

In this experiment, peptides were generated from a protein (APOL1) and the measurement of the peptides was used to evaluate whether the protein in the sample had a WT, G1 or G2 genotype. For example, as shown in FIG. 14, plasma punches were subjected to digestion (and extraction) with a protease (i.e., to generate genotype-specific peptides) and then the peptides were eluted from the strip analyzed by LC-MS/MS. It was found that plasma punches have less interference than blood punches (FIG. 15). The sample (i.e., filter paper punch outs comprising dried plasma) was added to digestion buffer (e.g., 0.675 mM dithiothreitol (DTT), 6.75 mg/mL deoxycholate (DOC), 50 mM Tris-HCL at pH 8.0±0.1.

After a short incubation at an elevation to denature proteins present in the sample, trypsin and an internal standard were added and digestion is allowed to proceed to generate analyte peptides. FIG. 16 shows a trypsin digestion time course for such analyte peptides using either liquid plasma or dried plasma in accordance with an embodiment of the disclosure. The formation of such surrogate peptide from ApoL1 (i.e., peptides 1-5) during digestion of liquid and dry plasma was evaluated in a time course analysis between 0 and 120 minutes. Samples of the liquid plasma digestion were collected at nine time points between 3 and 120 minutes. The profiles indicate digestion proceeds in a similar manner from both liquid and dry plasma specimens. Thus, there is virtually 100% digestion after about 2 hours.

Next formic acid was added to terminate the trypsin digestion and precipitate acid-insoluble materials (i.e., deoxycholate) and an aliquot of the supernatant was added to the LC-MS/MS system. FIG. 17 shows stability of peptides (i.e., peptides 1, 2 and 3) in dry plasma in accordance with an embodiment of the disclosure. It can be seen that the samples provide similar measured levels of the peptides even after 28 days at either 23 degrees Centigrade (the normal storage conditions) or 37 degrees Centigrade. The peptides that were characteristic of a particular allele (e.g., wild-type, G1 and/or G2) were then measured by LC-MS/MS to provide the sequence of the ApoL1 for allele specific peptides and the amino acid sequence of each of the allele specific peptides used to infer the ApoL1 genotype for the sample. It was found that proteomic genotyping using dried plasma vs liquid plasma biological samples gave identical results. When employed on a multiplexing LC system, such as the ARI Transcent™ TLX-4, injections may be run in parallel with injections staggered every 1.5 minutes to improve the duty cycle of the mass spectrometric analysis. Using this format, the time from tube to autosampler was 90 minutes and the overall procedure took about 260 minutes.

### Example 6

The disclosure may be better understood by reference to the following non-limiting embodiments.
A1. A method for determining the presence or amount of at least one biomarker of interest in a biological sample, the method comprising:
   providing a biological sample believed to contain at least one biomarker of interest wherein the biological sample comprises dried plasma;
   extracting the at least one biomarker of interest and at least one normalizing marker from the dried plasma into a liquid solution;
   measuring the at least one biomarker of interest by mass spectrometry;
   measuring the at least one normalizing maker by an analytical technique; and
   using the ratio of the measurements for at least one biomarker of interest and the at least one normalizing marker of interest to determine the presence or amount of the at least one biomarker of interest in the biological sample.
A2. The method according to any of the previous or subsequent embodiments, wherein the normalizing marker is endogenous to blood used for preparation of the dried plasma.
A3. The method according to any of the previous or subsequent embodiments, wherein the normalizing marker is present on the substrate used for preparation of the dried plasma.
A4. The method according to any of the previous or subsequent embodiments, wherein the normalizing marker has a CVg of less than 15.0%, or 10.0%, or 7.5%, or 5.0%.
A5. The method according to any of the previous or subsequent embodiments, wherein the normalizing marker comprises at least one of the normalizing markers listed in Table 1.
A6. The method according to any of the previous or subsequent embodiments, wherein an internal standard for the at least one biomarker of interest and/or the at least one normalizing maker is included in the liquid solution used for extracting the at least one biomarker of interest and at least one normalizing marker from the dried plasma into a liquid solution
A7. The method according to any of the previous or subsequent embodiments, wherein the internal standard is a stable isotope labeled analogue of the at least one biomarker and/or the at least one normalizing marker.
A8. The method according to any of the previous or subsequent embodiments, wherein at least one of a quality control or a calibrator (i.e. calibration standard) is added to the sample prior to extracting the at least one biomarker of interest and at least one normalizing marker from the dried plasma into a liquid solution.
A9. The method according to any of the previous or subsequent embodiments, wherein the liquid solution used for extraction comprises albumin prepared in water or aqueous buffer.
A10. The method according to any of the previous or subsequent embodiments, wherein the liquid solution used for extraction comprises water, aqueous buffer, non-aqueous solvent, or mixture thereof.
A11. The method according to any of the previous or subsequent embodiments, wherein the analytical technique used to measure the normalizing marker is mass spectrometry.
A12. The method according to any of the previous or subsequent embodiments, wherein the analytical technique used to measure the normalizing marker is an immunometric, colorimetric, fluorometric, immunoturbidimetric, and/or electrochemical method.
A13. The method according to any of the previous or subsequent embodiments, wherein the dried plasma is produced from blood deposited onto a sampling paper (e.g., laminar flow device) that provides separation of the plasma from the red blood cells.
A14. The method according to any of the previous or subsequent embodiments, wherein at least a portion of the liquid solution comprising the at least one biomarker and the at least one normalizing marker is subjected to a purification step prior to mass spectrometry.
A15. The method according to any of the previous or subsequent embodiments, wherein the purification comprises at least one of supported liquid extraction, liquid-liquid extraction, or protein precipitation of the sample or dilution of the sample prior to mass spectrometry.
A16. The method according to any of the previous or subsequent embodiments, wherein the purification step comprises chromatography.
A17. The method according to any of the previous or subsequent embodiments, wherein the chromatography comprises high performance liquid chromatography (HIPLC).
A18. The method according to any of the previous or subsequent embodiments, wherein the chromatography comprises extraction and analytical liquid chromatography.
A19. The method according to any of the previous or subsequent embodiments, wherein the at least one biomarker is analyzed by liquid chromatography tandem mass spectrometry (LC-MS/MS).
A20. The method according to any of the previous or subsequent embodiments, wherein the CVg of the normalizing marker is lower than the CVg of the biomarker of interest.
A21. The method according to any of the previous or subsequent embodiments, wherein the CVg of the normalizing marker is at least 10-fold, or 5-fold or 2-fold less than the CVg of the biomarker of interest.
B1. A system for determining the presence and/or amount of at least one biomarker in a biological sample, the system comprising:
   a device for providing a biological sample comprising dried plasma; and
   a station for analyzing the by mass spectrometry to determine the presence or amount of the at least one biomarker in the biological sample.
B2. The system according to any of the previous or subsequent embodiments, further comprising:
   a station for extracting the at least one biomarker and at least one normalizing marker from the dried plasma into a liquid solution;
   a station for measuring the at least one biomarker in the liquid solution by mass spectrometry;
   a station for measuring the at least one normalizing marker in the liquid solution; and
   a station for using the ratio of the measurements for the at least one biomarker and the at least one normalizing marker to determine the presence or amount of the at least one biomarker of interest in the biological sample.
B3. The system according to any of the previous or subsequent embodiments, further comprising a station to add an internal standard for the biomarker.
B4. The system according to any of the previous or subsequent embodiments, further comprising a station to add at least one of a quality control and/or a calibration standard for the biomarker.
B5. The system according to any of the previous or subsequent embodiments, further comprising a station for purification of the biomarker prior to mass spectrometry.
B6. The system according to any of the previous or subsequent embodiments, wherein the station for purification comprises a station to perform at least one of chromatography, supported liquid extraction, liquid-liquid extraction, or protein precipitation.
B7. The system according to any of the previous or subsequent embodiments, wherein the station for chromatography comprises high performance liquid chromatography (HPLC)
B8. The system according to any of the previous or subsequent embodiments, wherein the station for chromatography comprises extraction and analytical liquid chromatography.
B9. The system according to any of the previous or subsequent embodiments, wherein the device for providing a biological sample comprises a device to immobilize and separate red blood cells from plasma on a substrate.
B10. The system according to any of the previous or subsequent embodiments, further comprising a station for subjecting the sample proteolytic or chemical digestion.
B11. The system according to any of the previous or subsequent embodiments, wherein the station for mass spectrometry comprises a tandem mass spectrometer.
B12. The system according to any of the previous or subsequent embodiments, wherein the normalizing marker has a CVg of less than 5.0%.
B13. The system according to any of the previous or subsequent embodiments, wherein the normalizing marker comprises at least one of the normalizing markers listed in Table 1.
B14. The system according to any of the previous or subsequent embodiments, wherein at least one of the stations is controlled by a computer.

All documents referred to in this specification are herein incorporated by reference. Various modifications and variations to the described embodiments of the inventions will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

That which is claimed:

1. A method for determining an amount of at least one biomarker of interest in a plasma sample, the method comprising:
providing a sample of dried plasma believed to contain at least one biomarker of interest and containing at least one normalizing marker, wherein the at least one normalizing marker is endogenous to plasma used for preparation of the dried plasma;
extracting the at least one biomarker of interest and the at least one normalizing marker from the dried plasma into a liquid solution;
measuring the extracted at least one biomarker of interest by mass spectrometry;
measuring the extracted at least one normalizing marker by an analytical technique that is not mass spectrometry; and
using a ratio of the measured extracted the at least one biomarker of interest and the measured extracted the at least one normalizing marker to determine the amount of the at least one biomarker of interest in the plasma sample.

4. The method of claim 1, wherein the at least one normalizing marker has a coefficient of variability of a measurements (CVg) of less than 10.0%.

5. The method of claim 1, wherein the at least one normalizing marker comprises at least one of thyroxine binding globulin, sodium, chloride, calcium ions, lactate dehydrogenase 2, calcium, transferrin, total protein, serum albumin, sodium bicarbonate, or super oxide dismutase.

6. The method of claim 1, wherein an internal standard for the at least one biomarker of interest and/or the at least one normalizing marker is included in the liquid solution used for extracting the at least one biomarker of interest and at least one normalizing marker from the dried plasma into the liquid solution.

7. The method of claim 6, wherein the internal standard is a stable isotope labeled analogue of the at least one biomarker of interest and/or the at least one normalizing marker.

8. The method of claim 1, wherein at least one of a quality control or a calibrator is added to a substrate used for preparation of the dried plasma prior to extracting the at least one biomarker of interest and the at least one normalizing marker from the dried plasma into the liquid solution.

10. The method of claim 1, wherein the analytical technique used to measure the at least one normalizing marker is an immunometric, colorimetric, fluorometric, immunoturbidimetric and/or electrochemical method.

11. The method of claim 1, wherein the dried plasma is produced from blood deposited onto a laminar flow device that provides separation of the plasma sample from red blood cells.

12. The method of claim 1, wherein at least a portion of the liquid solution is subjected to purification prior to the measuring the at least one biomarker of interest by mass spectrometry.

13. The method of claim 12, wherein the purification comprises at least one of supported liquid extraction, liquid-liquid extraction, high turbulence liquid chromatography, or protein precipitation.

14. The method of claim 12, wherein the purification comprises HPLC.

15. The method of claim 1, wherein CVg of the at least one normalizing marker is lower than CVg of the at least one biomarker of interest.

16. The method of claim 1, wherein CVg of the at least one normalizing marker is at least 10-fold, or 5-fold or 2-fold less than CVg of the at least one biomarker of interest.

17. The method of claim 11, wherein the laminar flow device is a solid substrate.

18. The method of claim 11, wherein the solid substrate comprises filter paper or a membrane.

19. The method of claim 1, wherein the extracting is performed using a protein containing solution.

20. The method of claim 11, wherein the blood is heparinized blood.
